(19) United States
(12) Patent Application Publication
(10) Pub. No.: US 2025/0329415 A1
(43) Pub. Date: Oct. 23, 2025

(54) IDENTIFICATION OF SPLICING DISRUPTING MUTATIONS AND USE THEREOF

(71) Applicant: RAMOT AT TEL-AVIV UNIVERSITY LTD., Tel Aviv (IL)

(72) Inventors: Tamir TULLER, Tel Aviv (IL); Nicolas LYNN, Tel Aviv (IL)

(21) Appl. No.: 18/866,665

(22) PCT Filed: May 18, 2023
(86) PCT No.: PCT/IL2023/050515

§ 371 Date: Nov. 18, 2024

Related U.S. Application Data
(60) Provisional application No. 63/343,594, filed on May 19, 2022.

(51) Int. Cl. G16B 30/00; G16B 40/20; G16H 50/50

(52) U.S. Cl. CPC G16B 30/00; G16B 40/20; G16H 50/50

(57) ABSTRACT

Methods of identifying deleterious mutations and driver mutations comprising, identifying a mutation that disrupts or creates a splice donor or splice acceptor site and calculating a functional divergence score for the mutation wherein a score beyond a predetermined threshold indicates the mutation is a deleterious mutation are provided. Methods of evaluating or detecting cancer or a precancerous cell comprising identifying in genomic DNA mutations that disrupt or create a splice donor site or a splice acceptor site are also provided.

### CROSS REFERENCE TO RELATED APPLICATIONS

This application claims the benefit of priority of U.S. Provisional Patent Application No. 63/343,594, filed May 19, 2022, the contents of which are all incorporated herein by reference in its entirety.

### FIELD OF INVENTION

The present invention is in the field of cancer diagnostics.

### BACKGROUND OF THE INVENTION

Advancements in sequencing technology have made large collections of mutations and genomic information available through organizations including The Cancer Genome Atlas (TCGA), the Catalogue of Somatic Mutations in Cancer (COSMIC), and the 1000 Genomes Project. These datasets contain genomic information related to populations with a range of phenotypes, including cancer, and are often the product of Whole Exome Sequencing (WES) which provides profiles of variants found within a sample's protein-coding and protein coding-adjacent regions. Naturally, these datasets include millions of novel mutations that cannot all be experimentally studied due to numerous constraints.

Thus, most investigations that aim to characterize specific variants have focused their efforts on the analysis of select non-silent, non-synonymous mutations, or mutations that exist within the coding sequences (CDS) of genes and that alter the amino acid composition of encoded proteins through codon substitutions. Such a heuristic is effective in narrowing the search space to variants with a higher likelihood of having measurable effects. Yet, this strategy neglects millions of apparently silent mutations that also have functional—and potentially more severe—consequences. Silent and apparently silent mutations do not directly alter coding nucleotide sequences. Rather, they act on regulatory gene expression processes; they can exist within introns, untranslated regions, or even within CDSs if they result in synonymous codon exchanges and can hold strong predictive power in cancer classification and prognosis. Among the regulatory mechanisms that can be hijacked is splicing.

RNA splicing is a post-transcriptional modification step that transforms pre-mRNA sequences into mRNA transcripts. A single gene has multiple splicing blueprints, a phenomenon known as alternative splicing (AS). The most important cis acting elements needed for proper splicing include the 5′ intron boundary (acceptor-GU motif) and the 3′ intron boundary (donor-AG motif). However, there are also hundreds if not thousands of sequence determinants far within and beyond the intron that, while more difficult to characterize, play roles of varying importance in the decision of which GU/AG dinucleotides in the genome serve as functioning splice sites.

Ultimately, this means that cancerous apparently silent mutations could disrupt healthy gene expression by altering any of these countless splicing determinants. In doing so, those blueprints which define unique transcripts and healthy proteins can be reconfigured in a manner that is potentially more damaging than the replacement of a limited number of amino acids as is characteristic of missense mutations, for example. The same attribute that makes AS such a cost-effective method of introducing new proteins for evolutionary purification allows the wrong mutation to introduce disruptive alterations to existing proteins.

Estimates claim that 50% of human disease mutations cause splicing dysregulation. AS aberration has been detected in almost every major cancer-related phenomenon including angiogenesis, genomic instability, and apoptotic dysregulation. It was found that 68% of tumor samples contained at least one aberrant splicing-derived neoepitope while only 30% contained neoepitopes derived from somatic single-nucleotide variants, highlighting the increase in investigative targets that results from consideration of apparently silent oncogenic mechanisms. For example, it was shown that exons 4, 6, and 9 of TP53 contain functional hotspots for intron retention-caused inactivation by SNPs, and that mutations causing such effects are visible in lung squamous cell carcinoma (LUSC). In tumor suppressor gene (TSG) CDKN2A, a late base exonic mutation (LBEM) in exon 1 causing an intron retention resulted in complete inactivation of the protein. The Warburg effect, or the increased advantage of tumor cells to grow due to rapid energy generation through aerobic glycolysis, is dependent upon a shift in expression of pyruvate kinase (PKM) from adult splicing patterns (PKM1 isoform) to embryonic splicing patterns (PKM2 isoform). AIMP2-DX2 is an aberrantly spliced version of AIMP2, a strong TSG responsible for promoting programmed cell death, in which the second exon is deleted resulting in suppressed apoptotic activity in lung cancer. Switching between pro- and anti-angiogenic isoform of VDGFA is observed in cancer as well. Acquired drug resistance by tumors even has links to splicing, as was shown with a vemurafenib-resistant isoform of BRAF that is lacking exons 4-8. With respect to leveraging knowledge of aberrant splicing for cancer treatment, it was shown that reprogramming the splicing of BCL2L1 in tumor cells in favor of a pro-apoptotic variant—BCLXS—reduced tumor load in xenographs of metastatic melanoma. There is no shortage in examples that illustrate the impact of aberrant splicing in cancer progression and treatment potential, most of which are obtained from lab-based research. Unfortunately, one bottleneck to exploiting the splicing mechanism for driver identification is our inability to process and characterize millions of somatic mutations quickly and in a cancer type-independent manner.

Most work aimed at illuminating the roles of splicing in cancer approach the problem either from a reverse engineering perspective by assembling available RNA-seq data to attribute mutations with AS events, or with machine learning by building models that use splicing features to predict pathogenicity. Regarding the former, some investigations performed profiling of splicing aberration signatures found using NGS in prostate cancer cohorts while others develop useful web tools that illustrate splice isoforms found among cancer patients. Regarding the latter, IntSplice2, MMSplice, TraP, and S-CAP are tools employing neural networks, random forest models, or gradient boosting trees, generally function on variants within precise regions, and predict malignancy by training directly on clinical pathology annotations. However, to the best of our knowledge, there currently exists no tool that can quickly assess massive datasets of mutations and identify apparently silent cancer drivers as a secondary task based on predicted genomic and proteomic consequences, independent of cancer type, variant location, and a priori knowledge of pathogenicity. Such a tool is greatly needed.

### SUMMARY OF THE INVENTION

The present invention provides methods of identifying deleterious mutations and driver mutations comprising, identifying a mutation that disrupts or creates a splice donor or splice acceptor site and calculating a functional divergence score for the mutation wherein a score beyond a predetermined threshold indicates the mutation is a deleterious mutation. Methods of evaluating or detecting cancer or a precancerous cell comprising identifying in genomic DNA mutations that disrupt or create a splice donor site or a splice acceptor site are also provided.

According to a first aspect, there is provided a method of identifying a deleterious mutation in a cancer in a subject, the method comprising:
a. receiving mutation data from the cancer, wherein the mutation data comprises genomic sequence changes as compared to a healthy control genome;
b. selecting from the received mutation data a mutation that disrupts or creates a splice donor or splice acceptor site within a transcribed region;
c. for a selected mutation calculating all possible resultant spliced mRNA transcripts that can be produced from the transcribed region;
d. for all possible resultant spliced mRNA transcripts determining all possible amino acid sequence encoded; and
e. calculate a functional divergence score for the selected mutation based on the determined amino acid sequences as compared to a healthy control sequence, wherein the functional divergence score is a measure of the severity in protein function alteration present in the cancer as compared to a healthy control, and wherein a functional divergence score beyond a predetermined threshold indicates the selected mutation is a deleterious mutation;
thereby identifying a deleterious mutation in a cancer.

According to some embodiments, the cancer is selected from breast cancer, uterine cancer, head and neck cancer, brain cancer, prostate cancer, lung cancer, thyroid cancer, skin cancer, stomach cancer, bladder cancer, urothelial cancer, colon cancer, liver cancer, ovarian cancer, kidney cancer, cervical cancer, bone cancer, connective tissue cancer, esophageal cancer, pancreatic cancer, adrenal cancer, neuroendocrine cancer, rectal cancer, leukemia, testicular cancer, uveal cancer, bile duct cancer and lymphoma.

According to some embodiments, the received mutation data comprises whole exosome sequencing (WES) data from a sample comprising cancer DNA.

According to some embodiments, the sample is selected from a tumor sample and a bodily fluid sample, wherein the bodily fluid comprises cancer cells or cell free cancer DNA.

According to some embodiments, the healthy control genome is a consensus genome for species of which the subject is one or wherein the healthy control genome is a genome in a non-cancerous cell of the subject.

According to some embodiments, the received mutation data comprises mutations within exons, introns, and untranslated regions (UTRs).

According to some embodiments, a splice donor site comprises the sequence GU and a splice acceptor site comprises the sequence AG.

According to some embodiments, the selecting a mutation that disrupt or creates a splice donor or splice acceptor site comprises applying a trained machine learning algorithm to a genomic sequence comprising the mutation and wherein the trained machine learning algorithm outputs all predicted splice donor and splice acceptor sites affected by the mutation.

According to some embodiments, the trained machine learning algorithm is first applied to the genomic sequence without the mutation and the machine learning algorithm outputs all predicted splice donor and splice acceptor sties in the genomic sequence.

According to some embodiments, the machine learning algorithm outputs a probability score for a dinucleotide being a splice donor or splice acceptor site and wherein a site predicted to be affected by the mutation is a site whose score changes by at least a predetermined threshold from a probability score in the genomic sequence without the mutation to a probability score in the genomic sequence with the mutation.

According to some embodiments, the predetermined threshold is 690.

According to some embodiments, the genomic sequence comprises at least 10,000 nucleotides in addition to the mutation, optionally wherein the genomic sequence comprises at least 15,000 nucleotides in addition to the mutation.

According to some embodiments, the genomic sequence comprises at least 5000 nucleotides upstream of the mutation and at least 5000 nucleotides downstream of the mutation, optionally wherein the genomic sequence comprises at least 7500 nucleotides upstream of the mutation and at least 7500 nucleotides downstream of the mutation.

According to some embodiments, a mutation that disrupts a splice donor or acceptor site is a mutation that disrupts an annotated splice donor or acceptor site in the genome of the species of which the subject is one.

According to some embodiments, the calculating all possible resultant spliced mRNA transcripts comprises producing a list of all transcripts that can be created by linking a donor splice site to each downstream acceptor splice site that is present before the next donor splice site.

According to some embodiments, any transcript comprising a non-canonical exon comprising greater than 2000 nucleotides is discarded.

According to some embodiments, the determining the amino acid sequence encoded comprises determining all possible translation initiation sites (TIS) and from each TIS determining the amino acids encoded until a translation termination site (TTS) is reached.

According to some embodiments, the calculating a functional divergence score is based on a per residue evolutionary conservation values, and wherein divergence score is proportional or inversely proportional to the evolutionary conservation value of a residue present in the healthy control sequence and altered by the mutation.

According to some embodiments, a per residue evolutionary conservation value is calculated by a method comprising producing a multiple sequence alignment (MSA) from sequences of homologous proteins from different species and calculating a conservation value of each residue across the MSA.

According to some embodiments, the calculating a functional divergence score comprises calculating a deletion score comprising the sum of the per residue evolutionary conservation values for all residues not present in the determined amino acid sequence divided by the sum of all per residue evolutionary conservation values of the amino acid sequence, calculating an insertion score comprising the sum of the per residue evolutionary conservation values for all 4 amino acid residue blocks interrupted by an insertion divided by the sum of all per residue evolutionary conservation values of the amino acid sequence and multiplying the deletion score by the insertion score to produce a disruption score.

According to some embodiments, the functional divergence score is 1-the disruption score and beyond the predetermined threshold is below the predetermined threshold.

According to some embodiments, the predetermined threshold for said functional divergence score is 690.

According to some embodiments, the method comprises calculating a functional divergence score for all mutations that disrupt or create a splice donor or splice acceptor site.

According to some embodiments, the predetermined threshold is a bottom percentile of the mutations that produces the most functional divergence.

According to some embodiments, the percentile is the bottom 21st percentile of mutations by functional divergence score, wherein a lower score indicates greater divergence.

According to some embodiments, the calculating a functional divergence score comprises:
a. determining a functional divergence score for all determined amino acid sequences;
b. for each mRNA transcript averaging the functional divergence scores of all possible determined amino acid sequences; and
c. select the averaged functional divergence score indicating the greatest divergence as the functional divergence score for the mutation.

According to some embodiments, an identified deleterious mutation in a gene indicates the gene is a cancer driver gene in the cancer.

According to another aspect, there is provided a method of prognosing a subject suffering from cancer, the method comprising determining deleterious mutations in the cancer by a method comprising a method of the invention, wherein the number of deleterious mutations present is inversely related to the prognosis of the subject, thereby prognosing a subject suffering from cancer.

According to some embodiments, determining deleterious mutation comprises:
a. determining all deleterious mutation;
b. excluding mutations identified in control healthy subjects or tissue; or
c. a combination thereof.

According to some embodiments, the number of deleterious mutations is normalized to the total number of mutations in the cancer or the total number of mutations that disrupt or create a splice donor or splice acceptor site.

According to another aspect, there is provided a method of evaluating or detecting a cancer or precancerous cell in a subject, the method comprising:
a. receiving a sample from the subject comprising genomic DNA; and
b. identifying in the genomic DNA a mutation that disrupts or creates a splice donor or splice acceptor site within a gene selected from: AAAS, AASDH, AASS, ABCA12, ABCA2, ABCA8, ABHD1, ADAM8, ADAMTS20, ADAMTSL4, ADGRV1, ADNP, AGBL5, AGTPBP1, AHCTF1, AK9, AKAP12, AKAP3, ANKHD1, ANKRD12, ANKRD17, ANKRD31, ANKRD36C, ANKRD50, APC, APLP2, APOB, ARHGAP23, ARHGAP29, ARHGAP30, ARHGAP32, ARHGEF38, ARID2, ARID5B, ARMC5, ASPM, ATG2A, ATM, ATOSA, ATR, BAZIB, BAZ2A, BLM, BLTP2, BLTP3B, BOC, BRWD1, BTBD8, C15orf39, CAD, CCAR2, CCDC136, CCDC66, CCDC88A, CCDC88B, CCP110, CCPG1, CDHR4, CEP162, CEP250, CEP295, CFAP44, CHD6, CHD8, CHD9, CHRD, CIZ1, CLSPN, COL12A1, CSMD3, CTNND1, DCAF6, DCTN1, DDIAS, DHX8, DICER1, DIS3L, DMXL2, DNA2, DNAH10, DNAH12, DNAH14, DNAH2, DNAH7, DNAH8, DNAH9, DOCK5, DTHD1, DVL3, DYNC2H1, EDRF1, EIF3A, EIF4ENIF1, EPS8L2, ETAA1, EXPH5, FAM135A, FANCM, FBF1, FBXL5, FBXO11, FBXO38, FER1L5, FILIP1, FOXM1, FRMPD1, FRY, GFM2, GLI1, GNPTAB, GTF2I, GTF2IRD2, HECTD1, HECTD4, HIF1A, HLTF, HMGCR, IBTK, ICE2, IL17RC, IL6ST, INPP5F, INPPL1, IPO4, KAT6A, KCNH2, KIAA0232, KIAA0586, KIAA0825, KIAA2026, KIF23, KIF27, LAMA3, LAMB2, LARP1B, LCOR, LCORL, LMTK3, LOXHD1, LRIF1, LRP1, LRP2, LRRC9, LRRK2, LTN1, MAN2C1, MAP3K19, MAP4K4, MASTL, MCM7, MCM9, MDN1, MED1, MMRN1, MPDZ, MPHOSPH9, MSH2, MTMR4, MTOR, MYH13, MYH2, MYO15A, MYO9A, NCKIPSD, NCOR1, NF1, NIPBL, NLRX1, NOMO3, NPIPB4, NR3C1, NYAP1, ORC1, PBRM1, PCDH1, PDZD7, PELP1, PER3, PHF12, PHF3, PHLDB1, PHRF1, PIEZO1, PITPNM1, PKHD1, PLA2G2C, PLA2G2D, PLAA, PLAC8, PLAC9, PLCG1, PLEKHF1, PLEKHF2, PLEKHJ1, PLIN5, PLLP, PMP2, PMP22, PMS1, PNMT, PNOC, PNPO, PNRC1, POLE3, POLK, POLR1D, POLR2F, POLR2H, POLR2J2, POLR2K, POMC, POP5, POUIF1, PPCDC, PPCS, PPDPF, PPIG, PPIL3, PPM1M, PPM1N, PPP1R11, PPP6R1, PRDM1, PRDM11, PRICKLE1, PRPF40B, PRR30, PRR4, PRRT1, PRRT2, PRRT3, PRRT4, PRSS21, PRSS22, PRSS8, PRTN3, PSENEN, PSKH1, PSMA7, PSMB5, PSMB6, PSMC3IP, PSMD8, PSMD9, PSME1, PSME2, PSMG3, PSMG4, PSRC1, PTAR1, PTCRA, PTGDR, PTGER2, PTGIR, PTH, PTHLH, PTP4A1, PTP4A2, PTP4A3, PTPMT1, PTRH1, PTS, PUS1, PUS3, PWWP2A, PXMP2, PXN, PYCARD, PYCR1, PYCR2, PYGO2, QPRT, R3HDM1, R3HDM4, RAB11A, RAB11B, RAB11FIP2, RAB1A, RAB1B, RAB23, RAB24, RAB26, RAB29, RAB2B, RAB30, RAB33B, RAB34, RAB35, RAB3A, RAB3D, RAB40B, RAB40C, RAB4A, RAB4B, RAB5A, RAB5B, RAB5C, RAB8A, RABL2A, RAC1, RAC2, RAD1, RAD51, RAD9B, RAET1E, RALB, RALGAPA1, RALY, RAMP3, RANBP6, RAPH1, RARRES1, RASGRP2, RASGRP4, RASSF3, RASSF5, RASSF6, RASSF8, RAVER1, RBAK, RBCK1, RBFA, RBM12, RBM14, RBM15, RBM17, RBM22, RBM42, RBM43, RBM45, RBM47, RBSN, RCBTB1, RCBTB2, RCC1, RCC2, RCSD1, RDH12, RDM1, REG4, RELB, RELN, RERGL, REST, RFC3, RFT1, RFX5, RFX8, RGMA, RGMB, RGPD8, RGR, RGS17, RGS20, RGS4, RGS8, RHAG, RHBDD1, RHBDD2, RHBDL2, RHD, RHEB, RHOBTB1, RHOJ, RIC3, RIC8A, RIC8B, RILPL1, RIMKLB, RIN1, RMND5B, RNASEL, RNASET2, RND3, RNF114, RNF135, RNF138, RNF14, RNF141, RNF145, RNF182, RNF185, RNF19B, RNF2, RNF212B, RNF34, RNF41, RNF6, RNF8, RNH1, ROCK2, ROPN1, ROPN1B, RPA2, RPA3, RPL12, RPL14, RPL18, RPL27A, RPL37A, RPL4, RPL5, RPP14, RPP40, RPRD1A, RPS17, RPS21, RPS24, RPS3, RPS3A, RPS6KA4, RPUSD2, RRAS2, RREB1, RRM2, RRP8, RSBN1L, RSPH1, RSPH14, RSPH9, RYR3, SART3, SECISBP2L, SETD5, SGSM2, SHPRH, SIN3B, SKIC2, SLC12A4, SLC12A9, SMARCAD1, SMG7, SNX13, SNX14, SPEF2, SPEG, SPG11, SPTBN1, SRCAP, SSH1, SVEP1, SYCP2, SYNE2, SYNJ1, SYNM, SYNRG, SZT2, TDRD12, TJP1, TLR4, TNS2, TRRAP, TUT1, TYRO3, UACA, UBR4, UBR5, UNC79, UNC80, USH2A, USP33, USPL1, VCAN, VILL, VPS13C, VPS13D, WDR6, WIZ, YTHDC2, YY1AP1, ZBTB20, ZC3H6, ZC3H7A, ZCCHC2, ZFYVE16, ZHX1, ZHX3, ZMYM1, ZMYM6, ZNF208, ZNF226, ZNF268, ZNF280D, ZNF292, ZNF616, ZNF644, ZNF780B, ZNF814, ZNF841, and ZSCAN20;
thereby evaluating or detecting a cancer in a subject.

According to some embodiments, the evaluating comprises detecting a driver mutation in the cancer.

According to some embodiments, the identifying comprises sequencing the genomic DNA.

According to some embodiments, the sequencing is deep sequencing of next generation sequencing.

According to some embodiments, the sample is selected from a biopsy and a bodily fluid sample, wherein the bodily fluid comprises cells or cell free DNA.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description together with the drawings makes apparent to those skilled in the art how embodiments of the invention may be practiced.

FIG. 1: The outline of this investigation, beginning with variant identification and data parsing, then gene expression modeling of the variant's effects, followed by functional scoring, and finally validating mutation grades.

FIG. 2A-G: Reference dataset statistics. (2A) General dataset statistics across multiple variant descriptors show that the data passed to Onco-splice is highly diverse. (2B) The proportion of all unique mutations per variant type category indicates that most somatic mutations analyzed are SNPs. (2C) The proportion of all mutations per variant classification along with the retention of mutation in the mis-splicing and deleterious mis-splicing subsets; blue shades represent silent mutations and red shades represent non-silent mutations (splice region mutations occur within 3-8 bases of the intron or within 1-3 bases of the exon) reveals that most predicted deleterious mutations come from splice sites and regions, introns, and the ORF. SS-splice site, SR-splice region, SLT-silent, INTR-intron, IFD-in frame deletion, IFI-in frame insertion, MM-missense mutation, NM-nonsense mutation, TSS-translation start site, NS-nonstop mutation, FSI-frame shift insertion, FSD-frame shift deletion, 3UTR-3′ UTR, 5UTR-5′ UTR, 3FLK-3′ flank, 5FLK-5′ flank. (2D) The distribution of mutations per gene shows most genes have fewer than 2,000 identified variants across all patients. (2E) A breakdown of the cancer types analyzed and how many patients each project includes, with BRCA being the largest in terms of patient volume. (2F) The mean scores for mutations within each variant category. (2G) Distribution of Onco-splice scores across all analyzed mutations. BRCA: Breast invasive carcinoma, UCEC: uterine corpus endometrial carcinoma, HNSC: Head and neck squamous cell carcinoma, LGG: Brain lower grade glioma, PRAD: Prostate adenocarcinoma, LUAD: Lung adenocarcinoma, THCA: Thyroid carcinoma, SKCM: Skin cutaneious melanoma, STAD: Stomach adenocarcinoma, LUSC, Lung squamous cell carcinoma, BLCA: Bladder urothelial carcinoma, COAD: Colon adenocarcinoma, LIHC: Liver hepatocellular carcinoma, OV: Ovarian serous cystadenocarcinoma, KIRC: Kidney renal clear cell carcinoma, CESC: Cervical squamous cell carcinoma and endocervical adenocarcinoma, GBM: Glioblastoma multiforme, KIRP: Kidney renal papillary cell paraganglioma, READ: Rectum adenocarcinoma, LAML: Acute myeloid leukemia, TGCG: testicular germ cell tumors: THYM: Thymoma, ACC: Adrenocortical carcinoma, MESO: Mesothelioma, UVM: Uveal Melanoma, KICH: Kidney chromophobe, USC: Uterine carcinosarcoma, DLBC: Lymphoid neoplasm diffuse large B-cell lymphoma, CHOL-Cholangiocarcinoma.

FIGS. 3A-E: Architecture of Onco-splice. (3A) Overview of the steps taken in the pipeline to obtain a concise quantitative description of the functional loss that a mutation induces through predicted mis-splicing. (3B) A diagram illustrating the greedy approach to constructing transcript isoforms given only a pool of splice sites. (3C) Mature mRNA sequences are translated by selecting TISs with more optimal context based on TITER, Kozak context, and folding. (3D) Comparing two proteins using conservation scores per position using an algorithm that captures the loss due to insertions and deletions to the amino acid sequence. (3E) Aggregating functional loss scores for all transcripts in a gene using the weakest link method which assumes a mutation's pathogenic effects from its most disrupted transcript.

FIGS. 4A-C: (4A) As one filters de novo mutations into mis-splicing and deleterious mis-splicing subsets, one can see a depletion of null-occurring mutations, indicating that Onco-splice can differentiate between functional and benign variants that cause mild splicing aberrations; the depletion significance corresponding to healthy mutation depletion in the deleterious mis-splicing set is calculated by sampling from the mis-splicing set in an effort to isolate Onco-splice scores from SpliceAI. (4B) There is a significant difference in the scores assigned by Onco-splice to cancer-only and healthy-observed mutations, showing that the nature of aberrant splicing exhibited by each is distinct. (4C) ClinVar-overlapping mutations from the cancer cohort indicate that the variants classified as pathogenic have a significantly high ratio of pathogenic mutations compared to the set of mis-splicing mutations identified with SpliceAI or all the cancer-observed mutations.

FIGS. 5A-E: Pathogenicity predictor comparison. (5A) A tabular description of each alternative tool tested. (5B) Ratio of pathogenic, benign, and ambiguous variants found in ClinVar for subsets of predicted deleterious mutations as estimated using eight pathogenicity predictor's scores and recommended thresholds. (5C) ROC of different pathogenicity predictors shows that using this metric CADD offers the best performance. (5D) Correlations for scores generated by all tools indicate that some tools encode similar information while others do not. (5E) The positive predictive value of alternative pathogenicity predictors when scanning different thresholds.

FIGS. 6A-B: Pan-cancer driver enrichment. (6A) The hypergeometric p-value of the enrichment of known pan-cancer, TSG, and oncogene drivers across the top ranks of overrepresented genes shows that pan-cancer genes are better captured by Onco-splice scores. (6B) The hypergeometric p-value of the enrichment of known pan-cancer across genes that are overrepresented in mis-splicing and deleterious mis-splicing mutations across varying numbers of cancer types.

FIG. 7: The list of proposed cancer-related drivers is enriched for known cancer genes.

FIGS. 8A-F: (8A) The distributions of mutations per gene for the sets of all genes analyzed, canonical cancer drivers, and the proposed cancer genes show that the proposed genes come from the same distribution as the background gene set rather than having been selected based on trivial characteristics such as mutation volume. (8B) While the mutation volume for the proposed cancer drivers is not significantly different from all genes analyzed, the pathogenicity of the mutations found in these genes is significantly higher. (8C) Kaplan Meier survival probabilities for groups of patients defined using mutations within proposed cancer genes. (8D) Kaplan Meier survival probabilities for groups of patients defined using mutations within canonical cancer genes. (8E) Kaplan Meier survival probabilities for two groups of patients with similar mutation volumes segmented based on having or not having deleterious mutations. (8F) Distribution of mutation volumes for patients in groups identified in 8E shows that the patients do not have significantly different numbers of mutations.

### DETAILED DESCRIPTION OF THE INVENTION

The present invention, in some embodiments, provides methods of identifying deleterious mutations and driver mutations comprising, identifying a mutation that disrupts or creates a splice donor or splice acceptor site and calculating a functional divergence score for the mutation wherein a score beyond a predetermined threshold indicates the mutation is a deleterious mutation. Methods of evaluating or detecting cancer or a precancerous cell comprising identifying in genomic DNA mutations that disrupt or create a splice donor site or a splice acceptor site are also provided.

By a first aspect, there is provided a method of identifying a deleterious mutation in a cancer, the method comprising:
a. receiving mutation data from the cancer;
b. selecting from the received mutation data a mutation that disrupts or creates a splice donor or splice acceptor site;
c. for a selected mutation calculating all possible resultant mRNA transcripts that can be produced that comprise the mutation;
d. for all possible resultant mRNA transcripts determining all possible amino acid sequences encoded; and
e. calculate a functional divergence score for the selected mutation based on the determined amino acid sequences;
   thereby identifying a deleterious mutation in a cancer.

In some embodiments, the method is an in vitro method. In some embodiments, the method is an ex vivo method. In some embodiments, the method is a diagnostic method. In some embodiments, the method is a prognostic method. In some embodiments, the cancer is in a subject. In some embodiments, the cancer is from a subject. In some embodiments, the method is a method of diagnosing the subject. In some embodiments, the method is a method of prognosing the subject. In some embodiments, the method is a method of evaluating the cancer. In some embodiments, evaluating a cancer comprises estimating survival of the subject after diagnosis. In some embodiments, evaluating a cancer comprises determining the presence of cancer. In some embodiments, evaluating a cancer comprises evaluating a cancer's response to a therapeutic. In some embodiments, evaluating a cancer comprises evaluating a cancer's susceptibility to a therapeutic. In some embodiments, the evaluating is a companion diagnostic.

In some embodiments, evaluating a cancer comprises determining a driver mutation in the cancer. In some embodiments, a deleterious mutation is a driver mutation. In some embodiments, evaluating comprises determining a driver gene in the cancer. In some embodiments, evaluating a cancer comprises determining a disrupted pathway in the cancer. In some embodiments, a pathway is a signaling pathway. In some embodiments, disrupted is as compared to the pathway in a non-cancerous cell. In some embodiments, the non-cancerous cell is of the same cell type or tissue as the cancer.

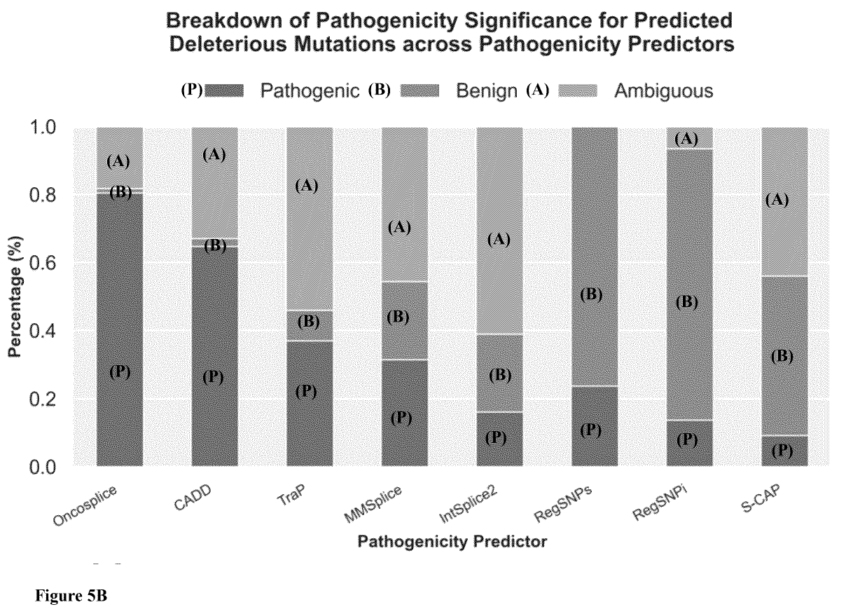

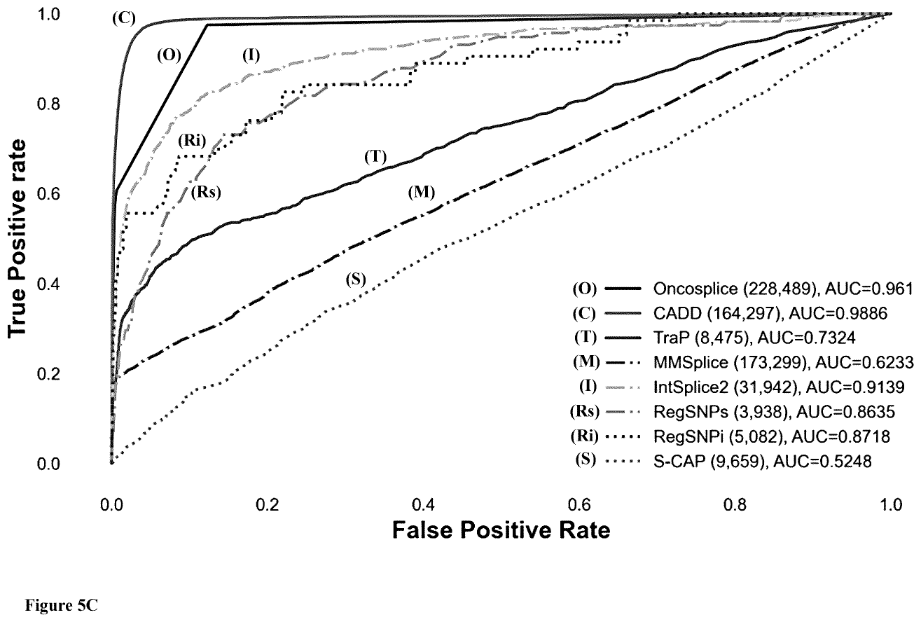

As used herein, the term “cancer” refers to a disease of cell proliferation. In some embodiments, cell proliferation is uncontrolled or overactive cell proliferation. In some embodiments, evaluating a cancer comprises determining the type of cancer. In some embodiments, the type of cancer is the tissue or cell type of origin of the cancer. In some embodiments, the cancer is a solid cancer. In some embodiments, the cancer is a hematopoietic cancer. In some embodiments, the type of cancer is a cancer type provided in FIG. 5. In some embodiments, the cancer type is selected from adrenal cancer, bladder cancer, urothelial cancer, breast cancer, cervical cancer, bile duct cancer, colon cancer, lymphoid cancer, esophageal cancer, brain cancer, head and neck cancer, renal cancer, liver cancer, lung cancer, mesodermal cancer, ovarian cancer, pancreatic cancer, endocrine cancer, neuroendocrine cancer, prostate cancer, rectal cancer, skin cancer, bone cancer, soft tissue cancer, stomach cancer, testicular cancer, thyroid cancer, uterine cancer and uveal cancer. In some embodiments, adrenal cancer is adrenocortical cancer. In some embodiments, adrenal cancer is pheochromocytoma. In some embodiments, cancer is carcinoma. In some embodiments, bladder cancer is bladder urothelial cancer. In some embodiments, breast cancer is breast invasive carcinoma. In some embodiments, the cancer is a squamous cell carcinoma. In some embodiments, the cancer is an adenocarcinoma. In some embodiments, the lymphoma is Lymphoid neoplasm diffuse large B-cell lymphoma. In some embodiments, the brain cancer is a glioma. In some embodiments, the glioma is glioblastoma. In some embodiments, the glioma is a low-grade glioma. In some embodiments, the kidney cancer is kidney chromophobe. In some embodiments, the kidney cancer is kidney renal clear cell carcinoma. In some embodiments, kidney cancer is kidney renal papillary cell carcinoma. In some embodiments, live cancer is liver hepatocellular carcinoma. In some embodiments, lung cancer is mesothelioma. In some embodiments, ovarian cancer is ovarian serous cystadenocarcinoma. In some embodiments, the neuroendocrine cancer is Paraganglioma. In some embodiments, bone cancer is sarcoma. In some embodiments, connective tissue cancer is sarcoma. In some embodiments, skin cancer is melanoma. In some embodiments, melanoma is skin cutaneous melanoma. In some embodiments, testicular cancer is testicular germ cell tumors. In some embodiments, thyroid cancer is thymoma. In some embodiments, uterine cancer is uterine corpus endometrial carcinoma. In some embodiments, the cancer is a carcinosarcoma. In some embodiments, the uveal cancer is uveal melanoma.

In some embodiments, the mutation data is genomic mutation data. In some embodiments, the mutation data comprises genomic sequences. In some embodiments, the mutation data is DNA sequence data. In some embodiments, the mutation data is data from a biopsy. In some embodiments, the biopsy is a cancer biopsy. In some embodiments, the biopsy is a tumor biopsy. In some embodiments, the biopsy is a liquid biopsy. As used herein, the term “liquid biopsy” refers from a blood sample from a cancer patient where cancer informative information can be isolated. In some embodiments, the cancer informative information is circulating tumor cells. In some embodiments, the informative information is cell free DNA (cfDNA). In some embodiments, the cfDNA is circulating tumor DNA (ctDNA). In some embodiments, the DNA sequence is sequences of cfDNA. In some embodiments, the mutation data is data from cfDNA. In some embodiments, the mutation data is data from cancer cells. In some embodiments, from cancer cells is directly from cancer cells. In some embodiments, cancer cells are cells in the tumor.

In some embodiments, the data comprises mutations. In some embodiments, the mutations are cancer mutations. In some embodiments, the mutations are from a cancer genome. In some embodiments, a cancer genome is a cancer cell genome. In some embodiments, a genomic sequence is a genome. In some embodiments, the genomic sequences are for a whole genome. In some embodiments, the mutations are all mutations in the genome. In some embodiments, the genomic sequences are from whole genome sequencing. In some embodiments, the genomic sequences are at least 1, 2, 3, 4, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 11000, 12000,13000, 14000 or 15000 sequences. Each possibility represents a separate embodiment of the invention. In some embodiments, the genomic sequences are a plurality of sequences. In some embodiments, sequences are locations. In some embodiments, sequences are genes. In some embodiments, a mutation is a DNA base or sequence that is different in the cancer as compared to a healthy control. In some embodiments, a healthy control is a healthy control genome. In some embodiments, a healthy control is a healthy control sequence. In some embodiments, the healthy control is an atlas of healthy genomic sequences. In some embodiments, the healthy control is a consensus sequence for the species of which the subject is one. In some embodiments, the consensus sequence is a consensus genome. Consensus genomes can be found for example in the NCBI genome browser and the UCSC genome browser. For example, for humans the GRCh38 human genome build can be employed. In some embodiments, the healthy control is a genomic sequence of a healthy individual. In some embodiments, the healthy control is a genomic sequence of a healthy tissue. In some embodiments, the healthy tissue is from the subject that suffers from the cancer. In some embodiments, the healthy tissue is from the subject that provided the genomic mutation data from the cancer. In some embodiments, the mutations are found in the cancer but are absent from healthy tissue of the subject. In some embodiments, the tissue is the same or of the same cell type from which the cancer originated. Thus, it will be understood by a skilled artisan that if for example the cancer is a lung cancer the mutation will not appear in the genome of healthy lung tissue from the subject. Similarly, if the cancer is a breast cancer or skin cancer the mutation would not appear in healthy breast or skin tissue, respectively, from the subject.

In some embodiments, a mutation is a point mutation. In some embodiments, a mutation is a deletion. In some embodiments, a mutation is an insertion. In some embodiments, a deletion is a deletion of 1 base. In some embodiments, a deletion is a deletion of 1, 2, 3, 4, or 5 bases. Each possibility represents a separate embodiment of the invention. In some embodiments, an insertion is an insertion of 1 base. In some embodiments, an insertion is an insertion of 1, 2, 3, 4, or 5 bases. Each possibility represents a separate embodiment of the invention.

In some embodiments, the mutation is in a gene. In some embodiments, the mutation is in a gene body. In some embodiments, the mutation is in a transcribed region. In some embodiments, the mutation is in a transcribed region that is translatable. In some embodiments, the mutation is in a transcribed region that can be translated to protein. In some embodiments, the mutation is in a transcribed region comprising an open reading frame encoding protein. In some embodiments, the mutation is in a transcribed region encoding a protein. In some embodiments, the mutation is in an open reading frame. In some embodiments, the mutation is in a region which is transcribed and spliced. In some embodiments, the mutation is in a region encoding an mRNA. In some embodiments, an mRNA is a pre-mRNA. In some embodiments, the mutation is a silent mutation. As used herein, the term “silent” mutation refers to all mutations that do not directly change a codon that codes for an amino acid into another codon that codes for another amino acid. In some embodiments, the mutation is not a non-synonymous mutation. In some embodiments, the genomic mutation data is devoid of non-silent mutations. In some embodiments, the mutation data is devoid of exonic non-synonymous mutations. In some embodiments, the mutation is a non-synonymous mutation. In some embodiments, the mutation data comprises exonic non-synonymous mutations.

The term “codon” refers to a sequence of three DNA or RNA nucleotides that correspond to a specific amino acid or stop signal during protein synthesis. The codon code is degenerate, in that more than one codon can code for the same amino acid. Such codons that code for the same amino acid are known as “synonymous” codons. Thus, for example, CUU, CUC, CUA, CUG, UUA, and UUG are synonymous codons that code for Leucine.

In some embodiments, the mutation is exonic. In some embodiments, the mutation is intronic. In some embodiments, the mutation is a synonymous mutation. In some embodiments, the mutation is in an untranslated region (UTR). In some embodiments, the UTR is the 5′ UTR. As used herein, the term “5′ UTR” refers to the sequence from the transcriptional start site of a gene until the translational start site. Thus, it is all of the 5′ sequence which is transcribed but not translated. In some embodiments, the UTR is the 3′ UTR. As used herein, the term “3′ UTR” refers to the sequence from the translational termination site to the transcriptional termination site. Thus, it is all of the 3′ sequence which is transcribed but not translated. It will be understood that the UTR is gene specific and that some genes have longer and some shorter UTRs. In some embodiments, the mutation is in a translated region.

In some embodiments, the mutation data is sequencing data. In some embodiments, the sequencing is deep sequencing. In some embodiments, sequencing is next generation sequencing (NGS). In some embodiments, sequencing is whole genome sequencing. In some embodiments, sequencing is whole exome sequencing (WES). In some embodiments, the method further comprises receiving sequencing data from the cancer. In some embodiments, the method further comprises receiving sequencing data from a non-cancerous tissue from the subject. In some embodiments, the non-cancerous tissue is the same tissue from which the cancer originated.

In some embodiments, from the cancer is from a sample. In some embodiments, the sample comprises cancer cells. In some embodiments, the sample comprises DNA. In some embodiments, the DNA is cancer DNA. In some embodiments, the sample is a tumor sample. In some embodiments, the sample is a biopsy. In some embodiments, the sample is a liquid biopsy. In some embodiments, the sample is a bodily fluid. In some embodiments, a bodily fluid is selected from blood, serum, plasma, gastric fluid, intestinal fluid, saliva, bile, tumor fluid, breast milk, urine, interstitial fluid, cerebral spinal fluid and stool. In some embodiments, the bodily fluid is blood or plasma. In some embodiments, the fluid is a fluid that contains cancer cells. In some embodiments, the fluid is a fluid that contains cell free DNA (cfDNA). In some embodiments, the cfDNA comprises cancer cfDNA. In some embodiments, the bodily fluid is selected from: blood, serum, plasma, gastric fluid, intestinal fluid, saliva, bile, tumor fluid, breast milk, urine, interstitial fluid, cerebral spinal fluid and stool. In some embodiments, the fluid is blood or plasma.

In some embodiments, the mutation disrupts a splice donor site. In some embodiments, the mutation disrupts a splice acceptor site. In some embodiments, the mutation creates a splice donor site. In some embodiments, the mutation creates a splice acceptor site. In some embodiments, the site is within a transcribed region. It will be understood by a skilled artisan that acceptor and donor sites are very short nucleotide sequences and such sequences produced outside a transcribed region are not relevant to the current method. In some embodiments, a splice donor site comprises the sequence GU. In some embodiments, a splice donor site comprises the sequence GURAGU. In some embodiments, a splice donor site comprises the sequence GGGURAGU. In some embodiments, a splice acceptor site comprises the sequence AG. In some embodiments, a splice acceptor site comprises the sequence NCAG. In some embodiments, a splice acceptor site comprises the sequence NCAGG.

In some embodiments, the splice acceptor site is downstream of a polypyrimidine tract. In some embodiments, a tract comprises at least 5, 6, 7, 8, 9, 10, 11, 12, 13, 14 or 15 pyrimidine bases. Each possibility represents a separate embodiment of the invention. In some embodiments, the pyrimidine passes are sequential. In some embodiments, the tract consists of the pyrimidine bases. In some embodiments, a tract comprises at least 15 bases. In some embodiments, the tract comprises between 15 and 20 bases. In some embodiments, downstream is at least 1 base downstream. In some embodiments, downstream is at least 1, 2, 3, 4, or 5 bases downstream. Each possibility represents a separate embodiment of the invention. In some embodiments, downstream is at most 30, 35, 40, 45, 50, 55, 60, 65, 70, 75, 80, 85, 90, 95, or 100 bases downstream. Each possibility represents a separate embodiment of the invention. In some embodiments, downstream is at most 40 bases downstream. In some embodiments, downstream is between 5 and 40 bases downstream. In some embodiments, the tract is downstream of branch sequence. In some embodiments, the branch sequence comprises the sequence YURAC. In some embodiments, the branch sequence is 20-100 nucleotides upstream of the splice acceptor site. In some embodiments, the branch sequence is 20-100 nucleotides upstream of the tract. In some embodiments, the branch sequence is 20-50 nucleotides upstream of the splice acceptor site. In some embodiments, the branch sequence is 20-50 nucleotides upstream of the tract.

In some embodiments, the mutation is a point mutation. In some embodiments, disrupting is mutating. In some embodiments, creating is mutating a no site sequence into a site sequence. In some embodiments, a mutation is a deletion. In some embodiments, disrupting is deleting. In some embodiments, deletion creates a site by the joining of the ends around the deletion. In some embodiments, a mutation is an insertion. In some embodiments, creating is inserting. In some embodiments, an insertion disrupts a site if the insertion occurs within the site.

In some embodiments, the mutation disrupts an annotated splice donor site. In some embodiments, the mutation disrupts an annotated splice acceptor site. In some embodiments, annotated is canonical. In some embodiments, annotated is in a genome. In some embodiments, the genome is a consensus genome. In some embodiments, the genome is from a species of which the subject is one. In some embodiments, the subject is a mammal. In some embodiments, the subject is a human. In some embodiments, a subject is in need of the method of the invention. In some embodiments, the subject suffers from cancer.

In some embodiments, selecting a mutation that disrupts or creates a site comprises applying a machine learning (ML) algorithm to a sequence comprising the mutation. ML algorithms that determine/identify splice sites are known in the art and any may be used. In some embodiments, the ML algorithm is SpliceAI. In some embodiments, the sequence is a genomic sequence. In some embodiments, selecting comprises employing a ML algorithm. In some embodiments, the ML algorithm is a trained algorithm. In some embodiments, the ML algorithm is a ML algorithm during training. In some embodiments, the algorithm is trained to predicted splice donor sites. In some embodiments, the algorithm is trained to predicted splice acceptor sites. In some embodiments, the algorithm is trained to predicted splice donor and splice acceptor sites. In some embodiments, predict is identify. In some embodiments, the ML algorithm is trained on a training set comprising sequences that are known to comprise splice donor and/or acceptor sites. In some embodiments, the training site comprises labels identifying a sequence as comprising a splice donor and/or acceptor site. In some embodiments, the labels identify the splice donor or acceptor site.

In some embodiments, the ML algorithm outputs predicted splice donor and/or splice acceptor sites. In some embodiments, the ML algorithm outputs predicted splice donor and/or splice acceptor sites affected by the mutation. In some embodiments, affected is disrupted or created. In some embodiments, the ML algorithm predicts all sites. In some embodiments, the ML algorithm predicts all effected sites. In some embodiments, the ML algorithm is applied to the sequence without the mutation. In some embodiments, the ML algorithm outputs predicted splice donor and/or splice acceptor sites in the sequence. In some embodiments, predicted sites is all predicted sites. In some embodiments, the ML algorithm is applied to the sequence without the mutation and to the sequence with the mutation and affected sites are selected. In some embodiments, selected sites are sites outputted only from the sequence with the mutation or only from the sequence without the mutation but not sites outputted from both sequences.

In some embodiments, the ML algorithm outputs a probability score. In some embodiments, the probability score is the probability of a sequence being a splice donor site. In some embodiments, the probability score is the probability of a sequence being a splice acceptor site. In some embodiments, the probability score is the probability of a sequence being a splice donor and/or acceptor site. In some embodiments, the sequence is a dinucleotide. In some embodiments, a sequence is a site. In some embodiments, a probability score is calculated for all dinucleotides in the sequence. In some embodiments, a sequence whose score changed by at least a predetermined threshold is a site predicted to be affected. In some embodiments, changes is changes from a probability score in the sequence without the mutation to a probability score in the sequence with the mutation. In some embodiments, a probability score that increases by more than a predetermined threshold is indicative of a created site. In some embodiments, a probability score that decreases by more than a predetermined threshold is indicative of a disrupted site. In some embodiments, the predetermined threshold is 0.5. In some embodiments, the predetermined threshold is a statistically significant change.

In some embodiments, the sequence to which the ML algorithm is applied is a genomic sequence. In some embodiments, the genomic sequence comprises at least 100, 250, 500, 750, 1000, 2000, 3000, 4000, 5000, 6000, 7000, 8000, 9000, 10000, 11000, 12000, 13000, 14000, 15000, 16000, 17000, 18000, 19000, or 20000 nucleotides in addition to the mutation. Each possibility represents a separate embodiment of the invention. In some embodiments, the genomic sequence comprises at least 1000 nucleotides. In some embodiments, the genomic sequence comprises at least 10000 nucleotides. In some embodiments, the genomic sequence comprises at least 15000 nucleotides. In some embodiments, the genomic sequence comprises at least 50, 100, 250, 500, 750, 1000, 1500, 2000, 2500, 3000, 3500, 4000, 4500, 5000, 5500, 6000, 6500, 7000, 7500, 8000, 8500, 9000, 9500, or 10000 nucleotides upstream of the mutation. Each possibility represents a separate embodiment of the invention. In some embodiments, the genomic sequence comprises at least 500 nucleotides upstream of the mutation. In some embodiments, the genomic sequence comprises at least 5000 nucleotides upstream of the mutation. In some embodiments, the genomic sequence comprises at least 7500 nucleotides upstream of the mutation. In some embodiments, the genomic sequence comprises at least 50, 100, 250, 500, 750, 1000, 1500, 2000, 2500, 3000, 3500, 4000, 4500, 5000, 5500, 6000, 6500, 7000, 7500, 8000, 8500, 9000, 9500, or 10000 nucleotides downstream of the mutation. Each possibility represents a separate embodiment of the invention. In some embodiments, the genomic sequence comprises at least 500 nucleotides downstream of the mutation. In some embodiments, the genomic sequence comprises at least 5000 nucleotides downstream of the mutation. In some embodiments, the genomic sequence comprises at least 7500 nucleotides downstream of the mutation.

In some embodiments, all possible mRNA transcripts are all possible pre-mRNA transcripts. In some embodiments, all possible mRNA transcripts are all possible unspliced mRNA transcripts. In some embodiments, all possible mRNA transcripts are all possible spliced mRNA transcripts. In some embodiments, all possible transcripts that comprise the mutation are all possible transcripts of the transcribed region. In some embodiments, all possible transcripts that comprise the mutation is all possible transcripts of the gene. In some embodiments, the gene is the gene comprising the mutation. It will be understood by a skilled artisan that more than one transcript can be generated for a genomic sequence. This may be due to alternative transcriptional initiation sites, alternative transcriptional termination sites, alternative promoters, alternative UTRs and alternative splicing (exon inclusion, exon exclusion, cryptic exons, etc.). In some embodiments, calculating all possible transcripts comprises all possible splice variants of the transcripts.

In some embodiments, calculating all possible spliced mRNA transcripts comprises producing a list of all transcripts that can be created by linking a donor splice site to each downstream acceptor site. In some embodiments, each downstream acceptor site is each downstream acceptor site that is before the next donor splice site. In some embodiments, the next donor splice site is the next annotated donor splice site. It will be understood that all possible splice variants are to be generated and considered while adhering to the rules of proper linkage in mRNA splicing. In some embodiments, a transcript comprising an exon of greater than 500, 600, 700, 750, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3500, 4000, 4500, or 5000 nucleotides is discarded. Each possibility represents a separate embodiment of the invention. In some embodiments, a transcript comprising an exon of greater than 2000 nucleotides is discarded. In some embodiments, the large exon is a non-canonical exon. In some embodiments, transcripts containing large canonical exons are retained.

In some embodiments, the method comprises calculating all possible pre-mRNA transcripts, calculating all possible spliced mRNA transcripts and calculated all possible amino acid sequences encoded. In some embodiments, from all pre-mRNA transcripts all possible spliced mRNA transcripts are calculated. In some embodiments, from all spliced mRNA transcripts all possible amino acid sequences encoded are calculated. In some embodiments, determining the amino acid sequence encoded comprises determining all possible translation initiation sites (TIS). In some embodiments, determining the amino acid sequence encoded comprises determining all possible translation termination sites (TTS). In some embodiments, determining the amino acid sequence encoded comprises determining the amino acids encoded from each TIS until each TTS. In some embodiments, all combinations of TIS to TTS are calculated. In some embodiments, determining the amino acid sequence encoded comprises determining all possible TIS and for each TIS determining the amino acids encoded until a TTS is reached.

In some embodiments, the functional divergence score is based on the determined amino acid sequences as compared to a healthy control sequence. In some embodiments, the functional divergence score is a measure of protein function alteration present in the cancer. In some embodiments, the functional divergence score is proportional to protein function alteration present in the cancer. In some embodiments, alteration is as compared to a healthy control. In some embodiments, healthy control is healthy control cells. In some embodiments, healthy control is healthy control tissue. It will be understood by a skilled artisan that the score indicates how greatly protein function has been affected. This value is determined without knowing what exact effect is produced. In some embodiments, a measure is a prediction. In some embodiments, a measure is an estimate.

In some embodiments, a functional divergence score beyond a predetermined threshold indicates the mutation is a deleterious mutation. In some embodiments, a functional divergence score beyond a predetermined threshold indicates the selected mutation is a deleterious mutation. In some embodiments, a functional divergence score is calculated as described hereinbelow. In some embodiments, a functional divergence score is calculated based on a per residue evolutionary conservation value. In some embodiments, a functional divergence score is proportional to the evolutionary conservation value of a residue present in the healthy control sequence and altered by the mutation. In some embodiments, a functional divergence score is inversely proportional to the evolutionary conservation value of a residue present in the healthy control sequence and altered by the mutation. In some embodiments, the predetermined threshold for the functional divergence score is 690.

In some embodiments, the predetermined threshold is the top percentage of mis-splicing mutations. In some embodiments, the top percent is the top 1, 2, 3, 4, 5, 6, 7, 8, 9, 10, 11, 12, 13, 14, 15, 16, 17, 18, 19, 20, 21, 22, 23, 24, or 25%. Each possibility represents a separate embodiment of the invention. In some embodiments, the top percentage is the top 5%. In some embodiments, the top percentage is the top 10%. In some embodiments, the top percentage is the top 25%.

In some embodiments, a per residue evolutionary conservation value is calculated. Methods and programs for calculating per residue evolutionary conservation and known in the art and any method/program may be used. In some embodiments, the program Rate4Site is used. In some embodiments, a per residue evolutionary conservation value is calculated by a method comprising producing a multiple sequence alignment (MSA). In some embodiments, the MSA is produced for protein encoded by the transcript. In some embodiments, the MSA is produced for the protein. In some embodiments, the MSA is produced for the protein encoded by the transcribed region comprising the mutation. In some embodiments, the MSA is produced for protein encoded by the sequence. In some embodiments, the MSA is a protein MSA. In some embodiments, amino acids residues are aligned in the MSA. In some embodiments, MSA is produced from sequences of homologous proteins from different species. Homologous protein sequences can be found in a variety of databases including the UCSC genome database. In some embodiments, a per residue evolutionary conservation value is calculated by calculating a conservation value of a residue in the MSA. In some embodiments, a per residue evolutionary conservation value is calculated by calculating a conservation value of each residue across the MSA. In some embodiments, the per residue value is normalized. In some embodiments, normalized is standardized. In some embodiments, normalized comprises dividing by the sum of the conservation values across the sequence.

In some embodiments, calculating a functional divergence score comprises calculating a deletion score. In some embodiments, a deletion score comprises the sum of the per residue evolutionary conservation values for all residues not present in the determined amino acid sequence divided by the sum of all per residue evolutionary conservation values of the amino acid sequence. In some embodiments, all residues not present are all deleted residues. In some embodiments, the sum of values of the deleted residues is divided by the sum of the values of all residues in the protein. It will be understood that the division by the values of the whole protein is done to normalize/standardize the values. This step ensures the score is between 1 and 0. In some embodiments, the deletion score is 1-the deletion score. Thus, if there are no deletions the deletion score will be 1.

In some embodiments, calculating a functional divergence score comprises calculating an insertion score. In some embodiments, an insertion score comprises the sum of the per residue evolutionary conservation values for a four amino acid residue block interrupted by the insertion divided by the sum of all per residue evolutionary conservation values of the amino acid sequence. In some embodiments, a four amino acid residue block comprises the two amino acids before the insertion and the two amino acids after the insertion. In some embodiments, a four amino acid residue block comprises one amino acid before the insertion and the three amino acids after the insertion or the three amino acids before the insertion and one amino acid after the insertion. In some embodiments, the sum of values of the four interrupted residues is divided by the sum of the values of all residues in the protein. It will be understood that the division by the values of the whole protein is done to normalize/standardize the values. This step ensures the score is between 1 and 0. In some embodiments, the insertion score is 1-the insertion score. Thus, if there are no insertions the insertion score will be 1.

In some embodiments, calculating a functional divergence score comprises multiplying the deletion score by the insertion score to produce a disruption score. If no deletions are present the disruption score will be equal to the insertion score. If no insertions are present the functional disruption score will be equal to the deletion score. In some embodiments, the functional divergence score is equal to the disruption score. In some embodiments, beyond the threshold is above the threshold. In some embodiments, the functional divergence score is equal to 1-the disruption score. In some embodiments, beyond the threshold is below the threshold. In some embodiments, the predetermined threshold is 0.327 (for a 1-disruption score). In some embodiments, the predetermined threshold is 690.

In some embodiments, the method comprises calculating a functional divergence score for all mutations that disrupt a splice donor site. In some embodiments, the method comprises calculating a functional divergence score for all mutations that disrupt a splice acceptor site. In some embodiments, the method comprises calculating a functional divergence score for all mutations that create a splice donor site. In some embodiments, the method comprises calculating a functional divergence score for all mutations that create a splice acceptor site. In some embodiments, the method comprises calculating a functional divergence score for all mutations that disrupt or create a splice donor or acceptor site. In some embodiments, the predetermined threshold is a bottom percentile of the mutations. In some embodiments, the bottom percentile is the mutations that produce the most functional divergence. In some embodiments, a lower score indicates greater divergence. In some embodiments, the predetermined threshold is a top percentile of the mutations. In some embodiments, the top percentile is the mutations that produce the most functional divergence. In some embodiments, a higher score indicates greater divergence. In some embodiments, a mutation within a predetermined percentile of disruption is indicated as a deleterious mutation. In some embodiments, the percentile that indicates a deleterious mutation is the 1, 2, 3, 4, 5, 6, 7, 8, 9, 10, 11, 12, 13, 14, 15, 16, 17, 18, 19, 20, 21, 22, 23, 24, or 25th percentile. Each possibility represents a separate embodiment of the invention. In some embodiments, the percentile that indicates a deleterious mutation is the 21st percentile. It will be understood that if the higher percentile indicates greater divergence, then the numbers will be the corresponding top percentiles and not bottom percentiles.

In some embodiments, calculating a functional divergence score comprises determining a functional divergence score for all determined amino acid sequences. In some embodiments, the method comprises averaging the functional divergence scores of all possible determined amino acid sequences for each mRNA transcript. In some embodiments, the method comprises selecting an averaged functional divergence score as the functional divergence score for the mutation. In some embodiments, the selected average score is the score indicating the greatest divergence. In some embodiments, the selected average score is the highest score. In some embodiments, the selected average score is the lowest score. Depending on the directionality of the score (whether a 1-conversion has been done) either the highest or lowest score will be selected.

In some embodiments, an identified deleterious mutation is a driver mutation. In some embodiments, an identified deleterious mutation in a gene indicates the gene is a cancer driver gene. In some embodiments, a driver is a driver in the cancer. In some embodiments, a driver is a driver for the subject. In some embodiments, a driver is used for evaluating the cancer. In some embodiments, a driver is used for prognosis.

According to another aspect, there is provided a method of prognosing a subject suffering from cancer, the method comprising determining deleterious mutations in the cancer by a method comprising a method of the invention, thereby prognosing a subject suffering from cancer.

In some embodiments, the number of deleterious mutations present is used for prognosis. In some embodiments, present is present in the cancer. In some embodiments, the number is proportional to the prognosis. In some embodiments, proportional is inversely proportional. In some embodiments, the number of deleterious mutations is normalized to the total number of mutations in the cancer. In some embodiments, the number of deleterious mutations is normalized to the total number of mutations in the cancer that disrupt or create a splice donor or splice acceptor site.

In some embodiments, determining deleterious mutations comprises determining all deleterious mutations. In some embodiments, all mutations excludes all mutations identified in a control healthy sample. In some embodiments, all mutations excludes all mutations identified in a control healthy subject. In some embodiments, all mutations excludes all mutations identified in a control healthy tissue.

According to another aspect, there is provided a method of evaluating or detecting a cancer or precancerous cell in a subject, the method comprising:
a. receiving a sample from the subject comprising DNA; and
b. identifying in the DNA a mutation that disrupts or creates a splice donor or splice acceptor site within a gene;
thereby evaluating or detecting a cancer in a subject.

In some embodiments, the gene is HSPE1, ACY1, MAF1, ATP6V1G1, ANAPC11, BAG2, ADM, APOF, TMEM170A, PPM1M, RPL34, NCF1, GPX4, SEC11A, RNF170, TMEM126B, CINP, CGREF1, CRIP3, ALG2, TMEM68, ZNF77, AUNIP, ARL9, ARL14EP, FUNDC1, PEF1, CGRRF1, CIDEC, GAPDH, NIPSNAP3B, DIO1, DAOA, COX7A2L, RBM11, AZI2, LYG2, STARD10, ARL1, SMPDL3A, MOB4, ATP6V0B, YEATS4, SURF1, LAPTM4A, RNF25, TMEM211, PRRG3, NT5DC2, FXYD4, DLK2, PCED1A, CENPT, RPS3A, STARD6, SLC25A36, TMEM161A, SLC16A5, OTUD6B, PSMA6, MAPK15, HEY1, DCUN1D2, ZNF445, CTSL, HOMER3, HPGD, RBMX2, GORASP1, RNASET2, ZNF254, UQCRB, KLRD1, AP3S1, ANKRD40, HAT1, TAF6L, LRWD1, UBA5, PPP2R2A, CCNC, ZMYND12, SPG21, BOLL, SLC36A4, ASB15, EXOSC9, FBXO3, BORA, SARAF, COPS4, HNRNPH3, SMPDL3B, ZNF43, SLC25A48, CELA1, UBE2U, TEKT1, TSNAXIP1, RAD51D, MOGS, CDC7, HTR3A, SMS, SEMA4F, ADA, ATF2, GGT7, ZMPSTE24, ARMC10, FAM104B, SLC7A8, MFSD9, CYP3A5, DPPA3, SLC38A2, EIF3M, ASIC5, HDC, MIER1, MTA2, CHEK1, PTPN9, RNF103, THOC1, ZNF527, DDX20, RPE65, SEC13, LANCL1, LHX9, DERA, SLC2A7, CREM, ATG16L2, LCORL, TMEM161B, ENTPD6, SCAMP5, UVRAG, B3GNTL1, TMEM120B, PRKRA, NEXN, CPNE9, ACSL3, KCTD3, TMC8, USP30, RBBP4, NSF, TLDC2, CRLF2, XRRA1, NAE1, LBP, ACADM, ABHD12, KANSL3, TRPC1, HEATR3, TESK2, CBX3, PTPN6, GSN, TUB, MTMR11, ARID3B, STRA8, NRG2, PTGR2, ERCC8, DYRK4, MFF, ADAMTSL4, CCHCR1, SKA3, MTMR14, TFAP2A, CRTAC1, DGKA, DOK5, ERN1, CCDC66, BAIAP2, CSNK2A1, IQCB1, INTS9, C7orf31, GRM6, PPM1B, GIT2, FAM135A, SETD5, PPARGC1A, AASS, HERC3, EMC1, GABRA3, NCAN, DNAI1, ZNF280D, CLCN5, TSPAN8, DDB1, PRRC2A, HSPD1, TGFBR3, EFCAB13, CYP2A13, LRSAM1, ARHGEF40, RADIL, MSH5, ROBO3, FMR1, NMD3, FIG4, EIF3A, CROT, OSBPL1A, WDR49, FTO, ARHGAP32, RPGRIP1L, AP4E1, SAMD12, KIAA0586, TDG, RBMX, TYRO3, CAD, TEX11, POLR3B, MCTP1, NNT, HLA-DRB5, ABCC1, SPTBN1, WWOX, PPFIA2, PRSS3, PAK2, HLA-DRB1, TJP1, ANKRD36, PLA2R1, NBPF12, ADAMTS20, MPDZ, CFAP47, ABCA12, MON2, SUPT6H, RICTOR, ABCA8, MTCH2, DOCK5, NBPF26, ATP2C1, SYCP2, RAPGEF4, HEATR5B, DOCK1, UNC80, SPEF2, LRRC7, or BDP1. Each possibility represents a separate embodiment of the invention. In some embodiments, the gene is AAAS, AASDH, AASS, ABCA12, ABCA2, ABCA8, ABHD1, ADAM8, ADAMTS20, ADAMTSL4, ADGRV1, ADNP, AGBL5, AGTPBP1, AHCTF1, AK9, AKAP12, AKAP3, ANKHD1, ANKRD12, ANKRD17, ANKRD31, ANKRD36C, ANKRD50, APC, APLP2, APOB, ARHGAP23, ARHGAP29, ARHGAP30, ARHGAP32, ARHGEF38, ARID2, ARID5B, ARMC5, ASPM, ATG2A, ATM, ATOSA, ATR, BAZIB, BAZ2A, BLM, BLTP2, BLTP3B, BOC, BRWD1, BTBD8, C15orf39, CAD, CCAR2, CCDC136, CCDC66, CCDC88A, CCDC88B, CCP110, CCPG1, CDHR4, CEP162, CEP250, CEP295, CFAP44, CHD6, CHD8, CHD9, CHRD, CIZ1, CLSPN, COL12A1, CSMD3, CTNND1, DCAF6, DCTN1, DDIAS, DHX8, DICER1, DIS3L, DMXL2, DNA2, DNAH10, DNAH12, DNAH14, DNAH2, DNAH7, DNAH8, DNAH9, DOCK5, DTHD1, DVL3, DYNC2H1, EDRF1, EIF3A, EIF4ENIF1, EPS8L2, ETAA1, EXPH5, FAM135A, FANCM, FBF1, FBXL5, FBXO11, FBXO38, FER1L5, FILIP1, FOXM1, FRMPD1, FRY, GFM2, GLI1, GNPTAB, GTF2I, GTF2IRD2, HECTD1, HECTD4, HIF1A, HLTF, HMGCR, IBTK, ICE2, IL17RC, IL6ST, INPP5F, INPPL1, IPO4, KAT6A, KCNH2, KIAA0232, KIAA0586, KIAA0825, KIAA2026, KIF23, KIF27, LAMA3, LAMB2, LARP1B, LCOR, LCORL, LMTK3, LOXHD1, LRIF1, LRP1, LRP2, LRRC9, LRRK2, LTN1, MAN2C1, MAP3K19, MAP4K4, MASTL, MCM7, MCM9, MDN1, MED1, MMRN1, MPDZ, MPHOSPH9, MSH2, MTMR4, MTOR, MYH13, MYH2, MYO15A, MYO9A, NCKIPSD, NCOR1, NF1, NIPBL, NLRX1, NOMO3, NPIPB4, NR3C1, NYAP1, ORC1, PBRM1, PCDH1, PDZD7, PELP1, PER3, PHF12, PHF3, PHLDB1, PHRF1, PIEZO1, PITPNM1, PKHD1, PLA2G2C, PLA2G2D, PLAA, PLAC8, PLAC9, PLCG1, PLEKHF1, PLEKHF2, PLEKHJ1, PLIN5, PLLP, PMP2, PMP22, PMS1, PNMT, PNOC, PNPO, PNRC1, POLE3, POLK, POLR1D, POLR2F, POLR2H, POLR2J2, POLR2K, POMC, POP5, POU1F1, PPCDC, PPCS, PPDPF, PPIG, PPIL3, PPM1M, PPM1N, PPP1R11, PPP6R1, PRDM1, PRDM11, PRICKLE1, PRPF40B, PRR30, PRR4, PRRT1, PRRT2, PRRT3, PRRT4, PRSS21, PRSS22, PRSS8, PRTN3, PSENEN, PSKH1, PSMA7, PSMB5, PSMB6, PSMC3IP, PSMD8, PSMD9, PSME1, PSME2, PSMG3, PSMG4, PSRC1, PTAR1, PTCRA, PTGDR, PTGER2, PTGIR, PTH, PTHLH, PTP4A1, PTP4A2, PTP4A3, PTPMT1, PTRH1, PTS, PUS1, PUS3, PWWP2A, PXMP2, PXN, PYCARD, PYCR1, PYCR2, PYGO2, QPRT, R3HDM1, R3HDM4, RAB11A, RAB11B, RAB11FIP2, RAB1A, RAB1B, RAB23, RAB24, RAB26, RAB29, RAB2B, RAB30, RAB33B, RAB34, RAB35, RAB3A, RAB3D, RAB40B, RAB40C, RAB4A, RAB4B, RAB5A, RAB5B, RAB5C, RAB8A, RABL2A, RAC1, RAC2, RAD1, RAD51, RAD9B, RAET1E, RALB, RALGAPA1, RALY, RAMP3, RANBP6, RAPH1, RARRES1, RASGRP2, RASGRP4, RASSF3, RASSF5, RASSF6, RASSF8, RAVER1, RBAK, RBCK1, RBFA, RBM12, RBM14, RBM15, RBM17, RBM22, RBM42, RBM43, RBM45, RBM47, RBSN, RCBTB1, RCBTB2, RCC1, RCC2, RCSD1, RDH12, RDM1, REG4, RELB, RELN, RERGL, REST, RFC3, RFT1, RFX5, RFX8, RGMA, RGMB, RGPD8, RGR, RGS17, RGS20, RGS4, RGS8, RHAG, RHBDD1, RHBDD2, RHBDL2, RHD, RHEB, RHOBTB1, RHOJ, RIC3, RICA, RIC8B, RILPL1, RIMKLB, RIN1, RMND5B, RNASEL, RNASET2, RND3, RNF114, RNF135, RNF138, RNF14, RNF141, RNF145, RNF182, RNF185, RNF19B, RNF2, RNF212B, RNF34, RNF41, RNF6, RNF8, RNH1, ROCK2, ROPN1, ROPN1B, RPA2, RPA3, RPL12, RPL14, RPL18, RPL27A, RPL37A, RPL4, RPL5, RPP14, RPP40, RPRD1A, RPS17, RPS21, RPS24, RPS3, RPS3A, RPS6KA4, RPUSD2, RRAS2, RREB1, RRM2, RRP8, RSBN1L, RSPH1, RSPH14, RSPH9, RYR3, SART3, SECISBP2L, SETD5, SGSM2, SHPRH, SIN3B, SKIC2, SLC12A4, SLC12A9, SMARCAD1, SMG7, SNX13, SNX14, SPEF2, SPEG, SPG11, SPTBN1, SRCAP, SSH1, SVEP1, SYCP2, SYNE2, SYNJ1, SYNM, SYNRG, SZT2, TDRD12, TJP1, TLR4, TNS2, TRRAP, TUT1, TYRO3, UACA, UBR4, UBR5, UNC79, UNC80, USH2A, USP33, USPL1, VCAN, VILL, VPS13C, VPS13D, WDR6, WIZ, YTHDC2, YY1AP1, ZBTB20, ZC3H6, ZC3H7A, ZCCHC2, ZFYVE16, ZHX1, ZHX3, ZMYM1, ZMYM6, ZNF208, ZNF226, ZNF268, ZNF280D, ZNF292, ZNF616, ZNF644, ZNF780B, ZNF814, ZNF841, or ZSCAN20. Each possibility represents a separate embodiment of the invention. In some embodiments, the gene is selected from PPM1M, RPS3A, RNASET2, LCORL, ADAMTSL4, CCDC66, FAM135A, SETD5, AASS, ZNF280D, EIF3A, ARHGAP32, KIAA0586, TYRO3, CAD, SPTBN1, TJP1, ADAMTS20, MPDZ, ABCA12, ABCA8, DOCK5, SYCP2, UNC80, and SPEF2. In some embodiments, the gene is PPM1M, RPS3A, RNASET2, LCORL, ADAMTSL4, CCDC66, FAM135A, SETD5, AASS, ZNF280D, EIF3A, ARHGAP32, KIAA0586, TYRO3, CAD, SPTBN1, TJP1, ADAMTS20, MPDZ, ABCA12, ABCA8, DOCK5, SYCP2, UNC80, or SPEF2. Each possibility represents a separate embodiment of the invention. In some embodiments, the gene is HERC3. In some embodiments, the gene is LHX9.

In some embodiments, the gene is selected from a gene provided in Table 1. In some embodiments, the DNA is genomic DNA. In some embodiments, the genomic DNA is circulating DNA. In some embodiments, evaluating comprises detecting a driver mutation. In some embodiments, evaluating comprises detecting a cancer driver gene. In some embodiments, identifying comprises sequencing. In some embodiments, sequencing is next generation sequencing. In some embodiments, sequencing is deep sequencing. In some embodiments, identification of the mutation indicates the presence of cancer. In some embodiments, identification of the mutation indicates the presence of a precancerous cell. In some embodiments, identification of the mutation indicates the presence of a cancer driver.

In some embodiments, the method further comprises treating the cancer. In some embodiments, the treating comprises administering to the subject an anticancer therapy. In some embodiments, the subject is the subject that provided the sample. In some embodiments, the subject is a subject suffering from cancer. In some embodiments, the subject is a subject in need of treatment. In some embodiments, the therapy is a therapeutic agent. In some embodiments, the therapy targets the determined driver gene. In some embodiments, the therapy targets another gene in a biological pathway comprising the driver gene. In some embodiments, the gene comprises a protein produced by the gene. Biological pathways are well known as are websites and programs for determining the biological pathways comprising a gene/protein and for performing pathway analysis. Such websites and programs include but are not limited to the Reactome Pathway Database (reactome.org), KEGG pathway database, Ingenuity Pathway analysis and Gene Ontology (GO) analysis. A skilled artisan will understand that though a mutation may exist in one gene it can be indirectly targeted by therapeutics against another gene/protein in the pathway (i.e., targeting a ligand with a therapeutic against its receptor, or targeting a protein in a complex with a therapeutic against other members of the complex).

In some embodiments, the therapy targets the determined driver mutation. In some embodiments, the therapy corrects the determined driver mutation. Methods of gene therapy and DNA correction are known in the art and any such method can be employed. Examples include CRISPR and other genome editing technologies, as well as antisense oligonucleotides (ASOs).

As used herein, the terms “administering,” “administration,” and like terms refer to any method which, in sound medical practice, delivers a composition containing an active agent to a subject in such a manner as to provide a therapeutic effect. Suitable routes of administration include oral, parenteral, subcutaneous, intravenous, intratumoral intramuscular, or intraperitoneal administration.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Methods

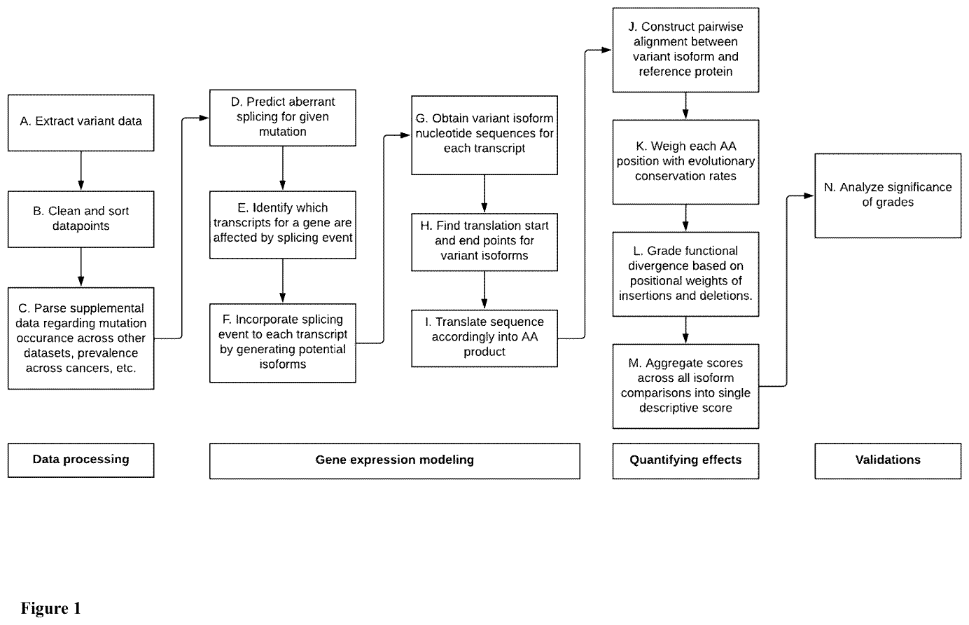

A schematic illustration that outlines this method procedure, beginning with variant identification and data parsing, then gene expression modeling of the variant's effects, followed by functional scoring, and finally validating mutation grades, is presented in FIG. 1.

### Data Preparation

Our primary data was aggregated from TCGA and includes 19.5M unique mutations within 16K genes found across 8,364 patients, each with one of 19 cancer types. The mutation types include single nucleotide polymorphisms (SNP), insertions (INS), and deletions (DEL), all scattered across intronic regions, splice sites, splice regions, and more.

Identifying Mis-Splicing Mutations with SpliceAI

The first step in Onco-splice is predicting mis-splicing events for each mutation. This is performed using SpliceAI, a deep residual neural network that confidently predicts splice site probabilities for each residue in a sequence based on 10,000 nucleotides of flanking context. The model is capable of splice-site identification with 95% top-k accuracy on arbitrary pre-mRNAs. SpliceAI is part of the module within Onco-splice that identifies changes to splice site usage. Whether a mutation causes aberrant splicing can be estimated using SpliceAI in tandem with reference genome annotations by tracking the changes in SpliceAI probabilities that nucleotides near a mutation experience. Given a mutation, if the donor or acceptor probability of a nearby site decreases by 0.5 or more and that same nucleotide is an annotated splice site, it is interpreted as a missed splicing event attributable to the respective mutation. If the donor or acceptor probability of a site increases by more than 0.5 and the nucleotide is not an annotated splice site, it is interpreted as a discovered splicing event. While it is possible for SpliceAI to detect splice sites that have not been formally annotated, there would be no sensible way to consider such junctions since the reference gene annotations do not include the position, and there would be no way to assess the quality of the prediction-hence they are ignored. The four detectable mis-splicing events include missed acceptors, missed donors, discovered acceptors, and discovered donors. Higher-order events, including mutually exclusive exons and intron retentions, are not the direct objectives.

Changes in splicing within a segment of 5,000 nucleotides around each mutation site (2,500 nucleotides upstream and downstream) were looked for. Each mutation is analyzed in isolation, regardless of other mutations that may also exist in the same gene and the same patient. 0.5 was used as a threshold for AS detection, which is validated in the original work and is the recommended SpliceAI parameter. Changes of this magnitude are rarely observed in randomized sequences.

### Modelling Variant Transcripts and Proteins

Each mutated gene considered by Onco-splice has reference genome annotations describing the blueprints for constructing its mature mRNA transcripts and proteins. This data is freely accessible, and annotations from the GENCODE database were used. Because SpliceAI does not consider the schema of all transcripts and donor-acceptor configurations that are biologically observed in each gene, it is not always obvious how splicing events can be incorporated into transcripts. Take, for instance, an adjacent canonical and predicted donor pair with no separating acceptor.

A greedy algorithm is used that operates on minimal assumptions to handle these situations. This method takes as input a pool of splice sites—reference and predicted alike—that reside within a pre-mRNA transcript's boundaries. The algorithm follows four rules:
1. Introduce and connect adjected nodes sequentially from 5′ to 3′.
2. Splice sites of the same type cannot be connected.
3. Adjacent splice sites of the same type are equal but exclusive options for connection continuation.
4. Generated splice paths must start with a donor and end with an acceptor.

These guidelines provide an effective construction strategy that is not dependent on unavailable experimental knowledge. The algorithm is not forced to create a single speculative isoform but can generate multiple possible mRNA transcript options. In fact, due to the dynamic and stochastic nature of splice site usage, many of the predicted variant transcripts may be produced, albeit at varying levels. This algorithm handles splice sites at the transcript level and does not require information regarding mutually exclusive exons, cassette exons, or alternative boundary usage. Once a mature mRNA transcript is defined, translation is modeled computationally. Greater detail in provided hereinbelow.

### Modeling Gene Expression

### Predicting Aberrant Splicing

Mutations at splice junctions (which disrupt essential GU/AG dinucleotides and necessarily result in a splice site deletion) that cause a change in SpliceAI probability of 0.5 or more validate in RNAseq at rate r, and all other non-splice site mutations causing a probability change above this threshold validate in RNAseq at ¾r when using this threshold

### Modeling Translation

Depending on the placement of a discovered site, the span of the transcript may be increased several times over, creating a very long, nonsensical exon. The biological likelihood of such an event occurring is quite low, and even in the case that it was generated by the splicing process, there would likely be some decay mechanisms that would suppress the lifespan of such abnormal transcripts. Transcript isoforms with novel exons longer than 2,000 nucleotides are discarded to account for this. This threshold was selected based on the knowledge that less than 1% of reference human-observed exons exceed 2,000 nucleotides in length.

After obtaining variant mature transcripts, the last major gene expression step is translation. Each transcript in the dataset contains one canonical translation initiation site (TIS) and one canonical translation termination site (TTS). Translating predicted mRNAs may seem trivial. However, untranslated region (UTR) boundaries available in reference transcript annotations may not be usable in variant transcripts. If a reference TIS is disturbed, then a new site is predicted using TITER, a deep learning model that predicts optimal TISs based on sequence context, as well as Kozak context score and RNA folding energy. In the case that the reference termination codon is interrupted, or an upstream frameshift renders it unusable, a new TTS is defined by finding the first in-frame canonical termination codon.

### Validations and Significance Testing

Various statistical testing methods were employed to validate the significance of the results. In the following sections, sample permutation testing and hypergeometric testing schemes are provided that are used recurrently. Additionally, scipy, an extensive statistical Python library, was employed to carry out χ², Mann-Whitney, Rank Sum, and ANOVA tests.

Validating using 1K Genome Project: To quantify the significance of the overlap between the mis-splicing mutation dataset and the null dataset, first the overlap is found, or the number of mutations in the mis-splicing subset that also occur in the null mutation set: N_{missplicing}^{null}. The total number of true mis-splicing mutations in the variant dataset is denoted as N_{missplicing}. The pool of all unique mutations observed in the full variant dataset is S_{unique}. For permutation testing, 1,000 iterations of the following procedure were performed:
1. Create a randomized subset of mutations by selecting N_{missplicing} mutations at random from S_{unique}. This is our fake, randomized subset of mis-splicing mutations.
2. For iteration i, N_{missplicing}^{fake} (i) is the quantity of mutations in the randomized mis-splicing mutation set that also occur in the null dataset.

The number of mis-splicing mutations expected to occur in the null dataset by chance is the mean of all N_{missplicing}^{fake} values. The p value of the true N_{missplicing}^{null} quantity is the number of iterations for which N_{missplicing}^{fake} is equal or smaller than N_{missplicing}^{null}, divided by the number of conducted iterations.

The hypergeometric probability of obtaining an equal or smaller overlap in null observed mutations within the mis-splicing subset is computed using the following equation:

$${P}_{hypergeometric} = {\sum }_{i = 0}^{{N}_{m issplicing}^{n u l l}} \frac{( \begin{matrix}{N}^{n u l l} \\ i\end{matrix} ) \cdot ( \begin{matrix}{N}_{u n i que} - {N}^{n u l l} \\ {N}_{u n i que} - {N}^{n u l l} - i\end{matrix} )}{( \begin{matrix}{N}_{u n i q u e} \\ {N}_{m issplicing}\end{matrix} )}$$

N_{missplicing}^{null}=number of mutations that are mis-splicing and in null set
N^{null}=number of null occurring mutations
N_{unique}=number of unique mutations in whole dataset
N_{missplicing}=number of mis-splicing mutation

Similar permutation and hypergeometric tests were performed when gauging the significance of null depletion in the deleterious mis-splicing subset, only differing in the set from which the random mutations are sampled (the depletion is tested relative to the mis-splicing subset in order to isolate the novel components without SpliceAI). Similar procedures are conducted several times across this investigation.

Validating with Clin Var

ClinVar data are parsed and binned into a set containing variant-identifying features (chromosome, mutation position, reference allele, and variant allele) along with their clinical significance and associated disease ontology terms. Clinical significance terms can take on several values though we retain only those with the following tags: “pathogenic”, “likely pathogenic”, “pathogenic/likely pathogenic”, “benign”, “likely benign”, “benign/likely benign”, “uncertain significance”, and “conflicting interpretations”. For simplicity, all values are grouped into “pathogenic” (terms 1-3), “benign” (terms 4-6), or “ambiguous” (terms 7-8) categories.

A joining operation is conducted between our unique cancer mutations and the ClinVar data on the variant-identifying features. This produces three distinct ClinVar associated variant sets: unique mutations, mis-splicing mutations, and deleterious mis-splicing mutations. For each subset the number of benign, ambiguous, and pathogenic variants were determined. The ratio of pathogenic to benign mutations was also calculated. The success of each subset is measured by the magnitude of this metric.

The significance associated with the pathogenic-to-benign ratio in the mis-splicing subset is defined by permutation testing; equally sized subsets of variants were randomized by sampling from all unique ClinVar-overlapping mutations and how many randomizations result in a pathogenic-to-benign ratio that is equal or greater is checked. The statistical significance associated with the deleterious mis-splicing subset is calculated similarly by sampling from the mis-splicing subset in order to isolate the power of Onco-splice novelties from SpliceAI's predictive power.

Comparing performance against other pathogenicity tools: The performance of Onco-splice was compared against seven alternative pathogenicity predictors, six of which are splicing-specific. To this end, pre-computed sets of mutations for CADD, S-CAP, TraP, and IntSplice2 were obtained. MMSplice, RegSNPs-Intron, and RegSNPs-Splicing did not have sets of pre-computed mutations available, so inference was performed on relevant subsets of the ClinVar dataset. The ROC for each tool was obtained using Python's sklearn library. The positive predictive value (PPV) for sets of mutations was obtained by taking all the true pathogenic variants among deleterious classifications and dividing that value by the size of the set of deleterious classifications. Correlations between any two tools were obtained by taking the subset of intersecting variants between those tools and finding the Pearson correlation between the scores of those variants. For tools that grade orthogonal variants, we see that there is no correlation value. For example, RegSNPs-Intron and RegSNPs-Splicing cannot grade the same variants; hence, no correlation is obtained.

Measuring cancer gene enrichment: To first obtain a baseline estimate as to whether cancer genes contain higher ratios of deleterious mutations compared to other genes, the significance of the average ratio of deleterious mutations to unique mutations was calculated across cancer genes and that value was compared to non-cancer gene ratios.

Permutation testing was employed by performing the following procedure 10,000 times:
1. For each gene g, calculate the rate of deleterious mutations as

$${R}_{g}^{del} = \frac{{N}_{g}^{del}}{{N}_{g}^{tot}}$$

where N_{g}^{del} is the number of deleterious mutations in g and N_{g}^{tot} is the number of total mutations in g.
2. Obtain the mean of all R_{g}^{del} for known cancer genes and call this R_{cancer}^{del}
3. Randomize a group of genes of size N_{cancer} where N_{cancer} is the number of known cancer genes used in Step 2.
4. Obtain the mean of the randomized gene group's R_{g}^{del} in iteration i, called random (i).

After performing these steps, determine how often these randomizations result in R_{random}^{del} that is greater than or equal to R_{cancer}^{del} by calculating:

$$p val = \frac{( {\sum }_{i}^{iterations} {R}_{r a n d o m}^{d e l} ( i ) \geq {R}_{c a n c e r}^{d e l} )}{iterations}$$

The objective is to validate Onco-splice's ability to identify cancer-driving mutations by showing that genes disproportionately overrepresented among deleterious mis-splicing mutations are enriched with known cancer genes. Yet, known cancer genes have more mutations than non-cancer genes and this bias must be addressed. Therefore, to find genes that are overrepresented by deleterious mutations while mitigating mutation volume bias, we design the following procedure which operates on any arbitrary pool of mutations.

The number of unique mutations for each gene—N_{unique} was determined. Based on this count, genes are divided into 5 quantile groups having similar mutation volumes.

For each gene, the count of mis-splicing (Nₘᵢₛ) and deleterious mis-splicing (N_{del}) mutations was determined and further these values were developed into mis-splicing and deleterious mis-splicing mutation ratios as:

$${R}_{m i s} = \frac{{N}_{m i s}}{{N}_{u n i q u e}} {R}_{d e l} = \frac{{N}_{d e l}}{{N}_{u n i q u e}}$$

Within each quantile group, genes are sorted based on one of the target ratios. To study, say, the top 5% of all overrepresented genes in the deleterious subset (as is done to identify the proposed set of novel cancer drivers), the top 5% of genes were select from each quantile based on R_{del}.

Once a set of overrepresented genes is obtained, the level of cancer gene enrichment can be obtained using permutation and hypergeometric testing as described previously. A similar strategy is followed when finding cancer-specific enrichment by performing this procedure on the sets of mutations found in each cancer type. The genes that are overrepresented in cancer type are tracked and then the total projects that each gene is found to be overrepresented in are counted.

### Estimating Patient Survival

To show the clinical value of the proposed cancer genes and Onco-splice two sets of patients were generated: one defined as the affected case set and one as the unaffected case set. In one survival analysis, the affected case set is determined by finding all the patients in the cohort who have one deleterious mutation in a defined set of cancer genes. The unaffected case set is determined by finding all the patients in the cohort who have no mis-splicing mutations in the same defined set of cancer genes. The set of cancer genes in the control experiment is defined as 375 known pan-cancer genes. The set of cancer genes in the variable experiment is defined as a random set of 375 genes from the proposed cancer gene set (375 genes were randomly sampled to ensure that there is no bias related to the size of the gene set). For each experiment (or set of affected and unaffected patients), the survival rates and the significance of their differences for 10- or 12-year survival were calculated using Kaplan Meier survival estimation. This analysis is robust to changes in the size of the gene set and the length of survival time. The significance of the test set is always stronger than the control set, regardless of the subset of 375 proposed cancer genes selected.

In a second survival analysis, the aim is to validate identified deleterious mutations while controlling for bias related to mutation volume in the selection of patients for each group. To this end, two sets of patients were generated: those who contain at least one gene affected by a deleterious mutation and those who are not affected by a deleterious mutation. These two sets of groups have a very strong difference in the distribution of mutation volumes, with the affected patients containing many more mutations than the unaffected case group. To understand if the signal persists when eliminating the mutation volume bias, subsets of patients that contain no significant difference in their distributions of mutation volumes are looked at by binning based on percentiles.

### Generating Consensus Cancer Gene Lists

At several stages in this investigation, canonical cancer drivers are used to validate and compare Onco-splice results. These reference cancer drivers are aggregated from various sources including COSMIC, the Network of Cancer Genes (NCG), the Tumor Suppressor Gene Database, the Oncogene Database, and more. In total, 591 pan-cancer driver genes, 224 of which have known TSG properties and 191 of which have known oncogenic properties, were identified. Additionally, 228 consensus cancer-specific genes that span all 19 cancer projects in this study were used.

### Identifying Gene Ontology (GO) Terms

Gene enrichment analysis was performed using g: Profiler, a web tool that performs hypergeometric enrichment analysis for a target gene set against a background gene set using a database of GO terms and their associated sets of terms. The primary list of genes was defined as the set of proposed novel cancer drivers. The background set is defined as all the genes with mutations that were studied. After running the analysis, g: Profiler provides adjusted p values for each identified term. This tool is updated with the latest GO terms and sets.

### Quantifying the Functional Divergence of Aberrant Proteins

Global pairwise alignment provides a good proxy for measuring the similarity between a healthy and predicted variant protein, such as those whose construction has been described. In the context of this investigation, a proper alignment must be selected carefully. In aberrant splicing, blocks of nucleotides are apparently inserted or deleted. This is considered by increasing the cost of opening gaps in the pairwise alignment while minimizing the cost of extending gaps. In principle, this prevents ad-hoc alignments with multiple illogical gaps and mismatches that serve only to maximize the alignment optimization. Biopython's pairwise alignment functionalities are used.

While effective, pairwise alignment is naïve since different amino acids in a protein are of varying importance. Certain residues play crucial roles in protein structure or function, and others are involved in neither. One way to ascertain the important domains in a protein is via evolutionary conservation, which uses the entropy observed for each amino acid residue in homologous proteins across species in the evolutionary tree as an estimate of functionality. Rate4Site—a probabilistic evolutionary conservation score calculator that uses Bayesian estimation to obtain relative mutation rates for each position in a multiple sequence alignment (MSA) of homologous proteins based on a phylogenic tree was used. To use Rate4Site, amino acid MSA files for 100 organisms relative to reference human proteins were obtained from UCSC. These MSA files were parsed and run through Rate4Site, generating a database of conservation vectors for thousands of proteins.

Using pairwise alignment, one can determine the exact positions that are deleted, inserted, and mismatched between the reference and variant protein. Using conservation scores, one can more accurately weigh each position's importance in the reference sequence. In calculating the magnitude of the functional effects of deletions and insertions, W was considered as a typical protein domain length. This value was obtained by taking the median of all functional domains across available proteins accessible through InterPro—75 amino acids. Dw is defined as the length of a detected deletion and Iw is defined as the length of a detected insertion. C (i, W) is the mean conservation score of a window of length W surrounding a position i in the protein.

$$C ( i , W ) = \frac{1}{W} \cdot {\sum }_{i - \frac{W}{2}}^{i + \frac{W}{2}} C ( j ) \qquad ( 1 )$$

C*(W) denotes the maximal mean conservation score of a window of length W in the analyzed protein. Let c (i, W) denote

$$\frac{C ( i , W )}{{C}^{*} ( W )} ,$$

the normalized and smoothed conservation vector.

$${C}^{*} ( W ) = {max}_{i} ( C ( i , W ) ) c ( i , W ) = \frac{C ( i , W )}{{C}^{*} ( W )} \qquad ( 2 )$$

Next, calculate the value of the deletion-derived functional loss for the deletion of Dw at position i as:

$${S}_{d e l} ( i ) = max ( 1 , \frac{D w}{W} ) c ( i , W ) \qquad ( 3 )$$

Then obtain the insertion-derived functional change for the deletion of iW at position i as:

$${S}_{i n s} ( i ) = max ( 1 , \frac{Iw}{W} ) \cdot c ( i , W ) \qquad ( 4 )$$

The total penalty for all the deletions and insertions observed in a particular protein is computed using a sliding window of size W conflating across deletion and insertion penalties as follows:

$$S ( i ) = {\sum }_{i - \frac{W}{2}}^{i + \frac{W}{2}} {S}_{d e l} ( j ) + {S}_{i n s} ( j ) \qquad ( 5 )$$

The final score for the respective protein comparison is taken as the maximum value of the penalty vector.

$${S}_{p athogenicity} = {max}_{i} S ( i ) \qquad ( 6 )$$

### Aggregating Scores Across Transcripts and Variant Libraries

A gene is responsible for multiple functionalities, each characterized by its transcripts. If even a single transcript is dysfunctional, pathogenesis may occur. When analyzing a library of products for a mutated gene without knowledge of the roles of each protein, one may be more interested in how dysfunctional the most negatively affected transcript for that mutated gene is. A simple average across all modeled transcripts for a gene could dilute the negative impact of a single poorly preserved transcript if the others are all unaffected by an aberrant splicing event.

To address this, the weakest-link strategy was implemented which obtains the average score for each transcript of a mutated gene across all its predicted isoforms and then assigns the highest score across those transcripts to the mutation. This strategy describes a mutation by the most dysfunctional protein it generates.

### Results

### Example 1: Approximately 1.3% of all Somatic Mutations in Cancer Patients are Predicted to Cause Aberrant Splicing

A dataset containing 12.25M unique somatic mutations within 9,879 protein-coding genes (for which we have adequate evolutionary conservation coverage) found across 8,364 patients from the TCGA catalog was examined. Germline mutations were not considered. The mutations accessed were filtered based on quality tests conducted by the dataset authors and have mean allele frequencies (MAF) lower than 0.01 and as high as 0.74 within the healthy population. These mutations are found using WES, a sequencing procedure that targets CDSs. Only partial identification of intergenic and deep intronic variants is expected due to the dependence on WES. However, this analysis will not be harmed by undetected mutations because unique mutations are analyzed in isolation rather than the ensemble of all mutations found within a gene and patient. The variant types available include single nucleotide polymorphisms (SNP), insertions (INS), and deletions (DEL), all scattered across intronic regions, splice sites, coding regions, and more.

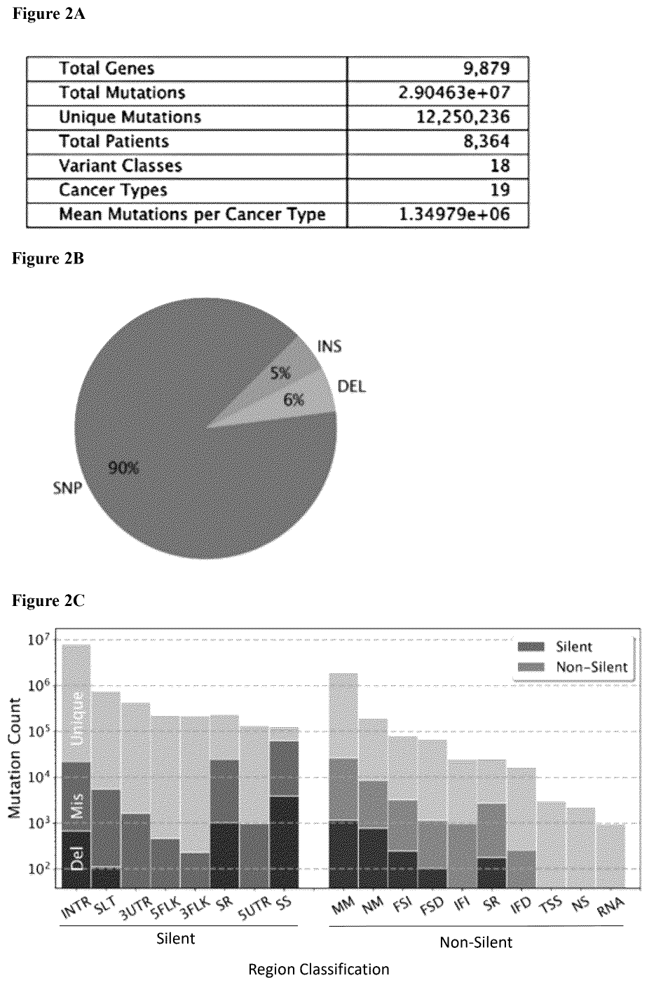

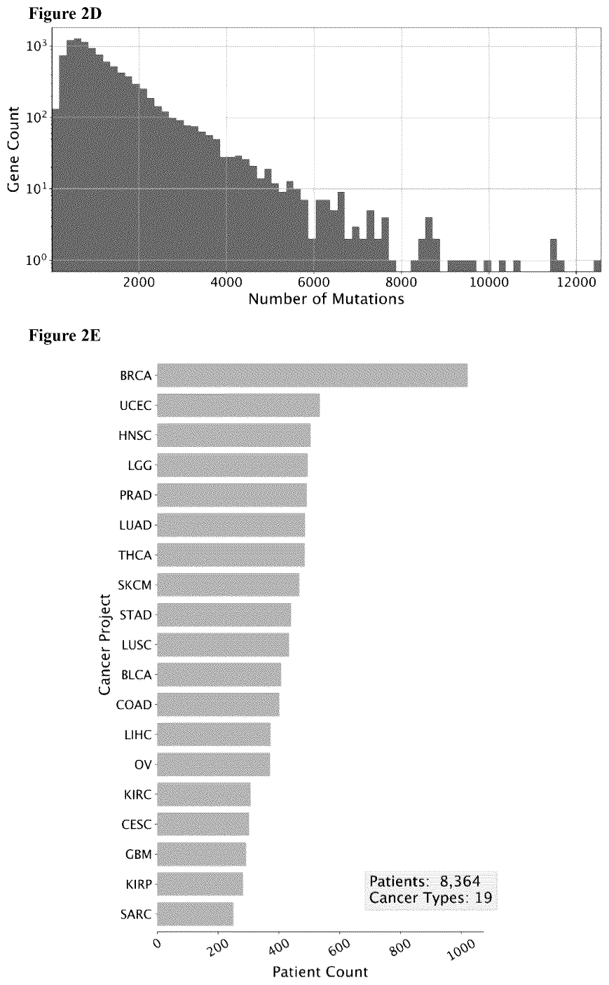

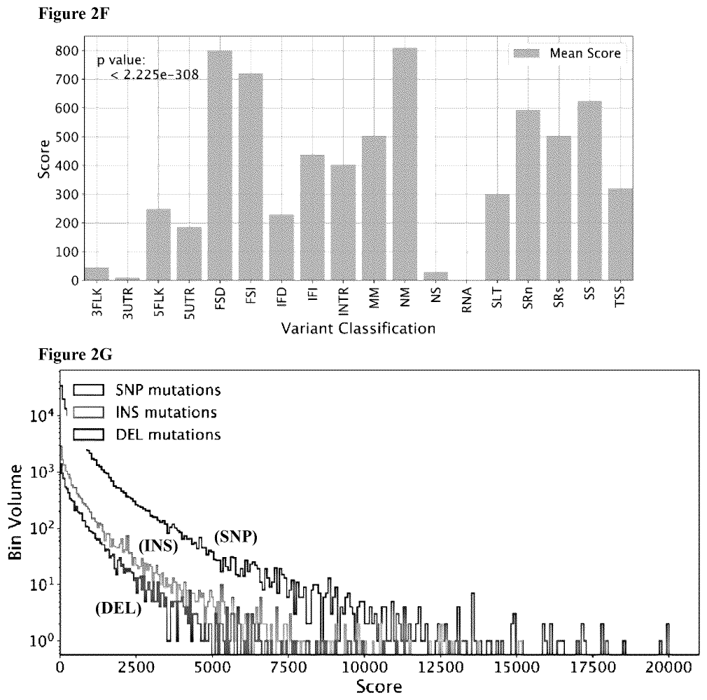

Out of all somatic mutations graded with Onco-splice (the method of the invention), roughly 159K (1.3%) are predicted to result in aberrant splicing, henceforth referred to as mis-splicing mutations. All mis-splicing mutations were used to model predicted aberrant sequence outcomes. While experimental sequencing data to validate the proteomic and transcriptomic predictions for each mutation are unavailable, Onco-splice's scores can be used (FIG. 2F-G), which estimate the functional difference between two proteomes, to determine if Onco-splice models capture meaningful signals. The top 5th percentile of variants based on Onco-splice grades accounts for 8.2K mis-splicing mutations, or 0.067% of all unique mutations analyzed, and represents variants with raw grades of at least 2,000; such mutations will be referred to as deleterious mis-splicing mutations and represent variants classified as pathogenic using the Onco-splice divergence scores. This cutoff was selected based on optimization of PPV and will be discussed further. FIGS. 2A-E show a dimensional breakdown of the diverse reference dataset tested.

As expected, almost all splice site mutations are predicted to result in a mis-splicing event (specifically, the deletion of the corresponding splice site). Around 39% of mis-splicing mutations and 47% of deleterious mutations are identified as splice site mutations. More interestingly, however, is that 16% of predicted mis-splicing mutations are made up of missense variants, as seen in FIG. 2C. This indicates that many previously investigated non-silent mutations may have secondary consequences related to splicing past their distracting amino acid exchanges.

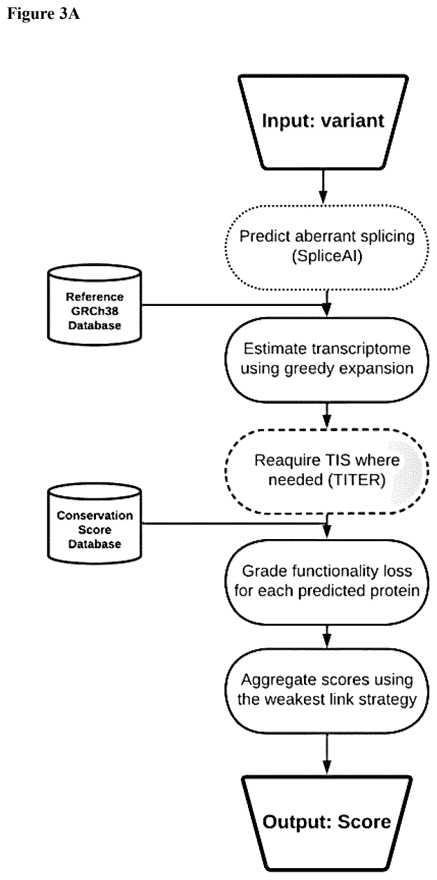

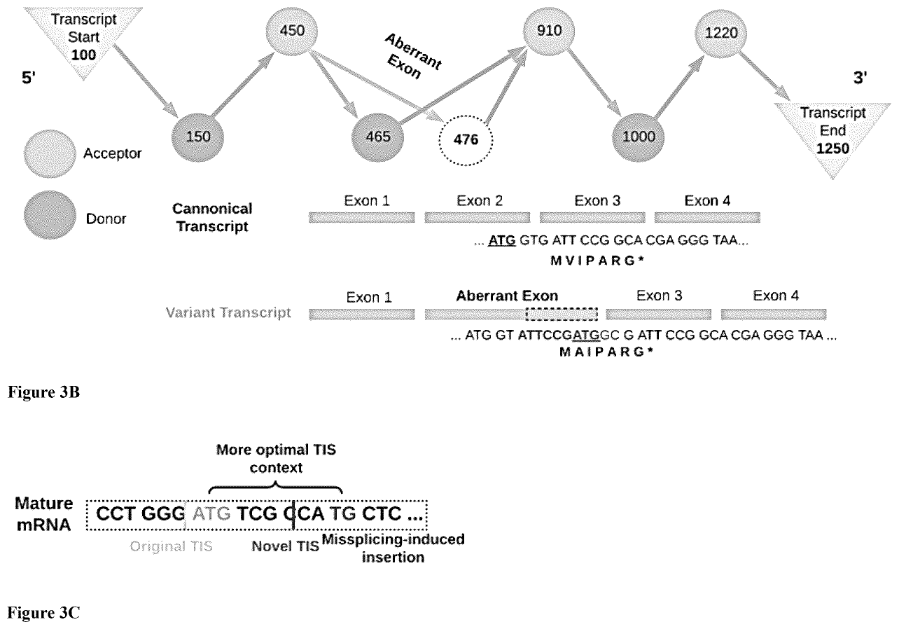

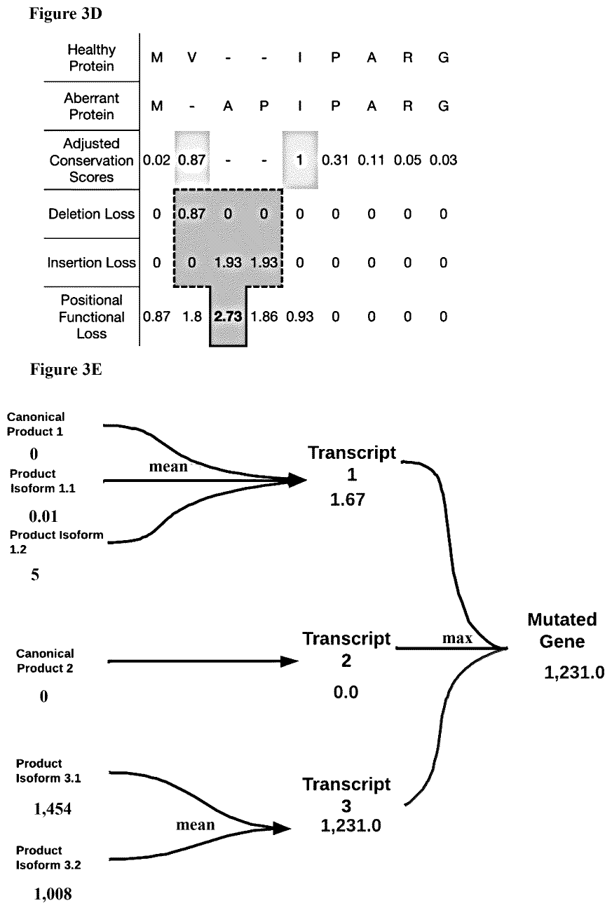

Onco-splice assigns scores to each mis-splicing mutation using the mechanism illustrated in FIGS. 3A-E. These scores quantify the decrease in similarity—and thus decrease in functionality—between corresponding healthy and variant proteins resulting from splicing aberration. Scores range between zero and one, where the former indicates the most severe disruption of a resulting protein, and the latter indicates no measurable difference.

The scores for all mutations across each variant type can be seen in FIGS. 2F-G. The relatively stable distribution of grades indicates that mutations affecting splicing range in predicted consequences. Additionally, this stability allows for grouped analysis, rather than requiring that we conduct observations on each variant type individually. There is an observable excess of one-scoring mutations which comes from detected splice site events in transcripts whose ORF is not affected (such as splice site changes in UTR regions which our tool is not yet capable of scoring) or from variants affecting splice sites in a transcript which is not available in our mRNA dataset (such as a discovered splice site too far from all documented transcripts). It can also be seen that there are very few mutations with grades of zero since some alignment between a reference and variant amino acid sequence is always possible, though we expect that once this alignment falls past a critical point, the protein is dysfunctional.

### Example 2: Deleterious Mis-Splicing Mutations are Significantly Depleted within the Healthy Population and Correlate Highly with Clinically Identified Pathogenic Variants

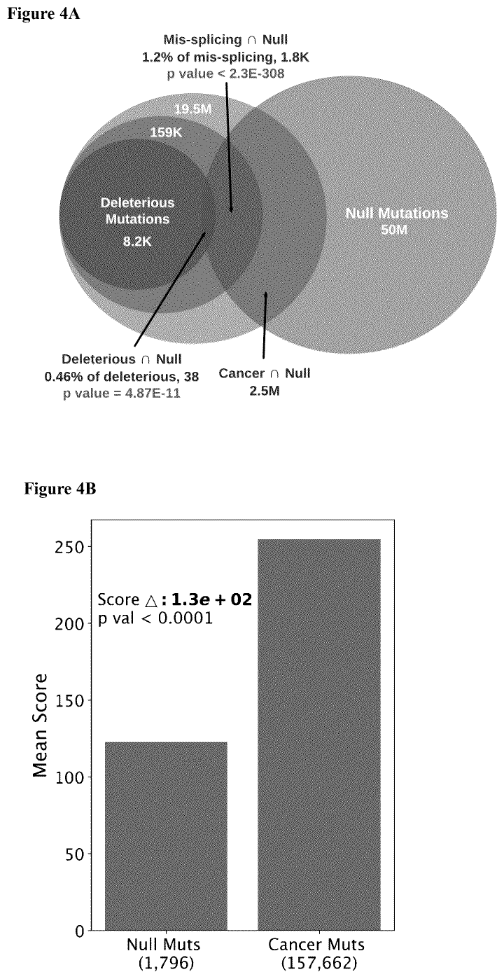

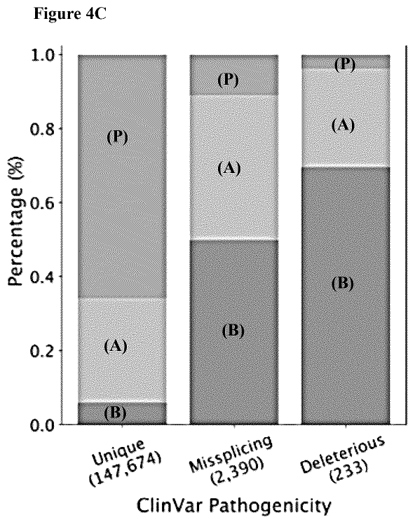

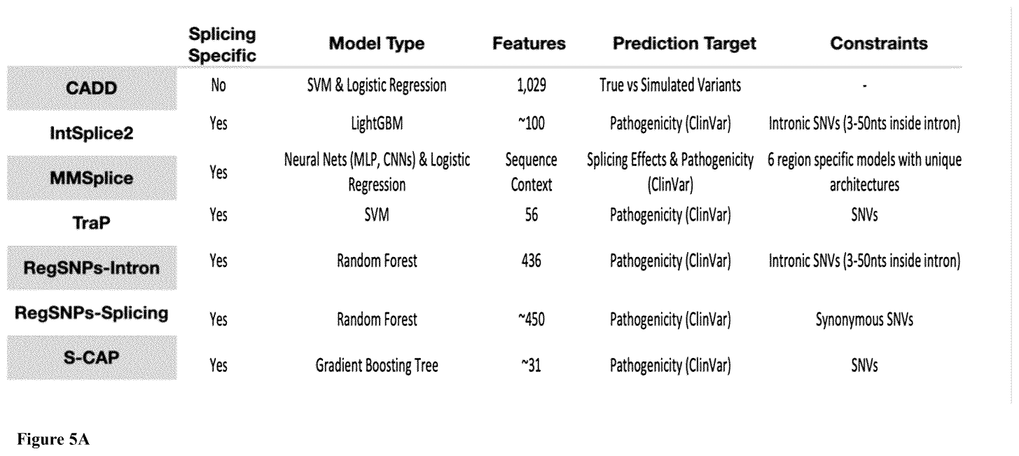

A set of 50M mutations was obtained from the 1000 Genome Project which holds variants observed among more than 2.6K diverse individuals. The variants present in this cohort have frequencies of at least 1% within their respective healthy populations. Conservative assumptions were adopted; mutations are considered benign if they occur within this reference database, though one expects that some mutations found within the general population can also be deleterious. In this set, 2.5M variants intersect with the cancer-associated mutations. These overlapping variants are diverse across all descriptors. An indication that Onco-splice scores are meaningful would be a depletion of healthy-occurring variants among mis-splicing and deleterious mis-splicing subsets, a concept illustrated in FIG. 4A.

159K cancer-observed mis-splicing variants were identified. Of those, only 1.8K or 1.13% are seen in the healthy population (permutation test mean: 32,014, permutation p-value: <0.001, hypergeometric p-value: <2.3E-308 Chi-square <2.3E-308) indicating that SpliceAI can detect aberrant splice-inducing mutations and that these mis-splicing mutations are more frequent in cancer patients than in the healthy population. 8.2K deleterious mis-splicing mutations were further identified and it was found that only 38 or 0.46% are observed in the healthy population (permutation test mean: 92, permutation p-value: <0.001, hypergeometric p-value: 4.87E-11, Chi-square: 1.63E-8; FIG. 4A.), a strong depletion relative to the mis-splicing mutation set which implies that Onco-splice scores contribute significant additional information past checking for aberrant splicing.

By further leveraging the healthy-occurring mutations one can see that cancer-associated mis-splicing mutations receive more pathogenic scores than healthy-observed mis-splicing mutations (difference: 132, permutation random mean: −0.002, p-value: <0.0001, Wilcoxon Rank Sum: 8.66E-83) as shown in Error! Reference source not found.B. Since it is expected that mis-splicing mutations in the healthy population would generally have less severe disease-related effects, this further suggests that Onco-splice scores accurately convey the nature of a variant's functional consequences. Onco-splice scores are not interpretable as probability values and are better used for comparing changes to function. To reiterate, we expect many if not a majority of cancer-observed mutations to be benign and some healthy-observed mutations to be deleterious. Despite this noise, the difference in score between the two large, unannotated sets of variants clearly illustrates that cancer-associated mutations cause more deleterious mis-splicing events than those observed in the healthy population, even when heavily diluted by many benign variants.

While pathogenicity ground truths are unavailable for most de novo mutations, there are some sources that aggregate clinical associations for sizable sets of variants such as ClinVar. 1.1M ClinVar mutations were downloaded to investigate any overlap they may have with the working dataset. Of those, 148K mutations intersected with the current cancer-observed dataset. Moreover, 2.4K of those mutations result in a predicted mis-splicing event while 233 also result in deleterious forms of mis-splicing. If Onco-splice grades properly describe pathogenicity, a greater concentration of clinically verified disease-associated mutations should be observed in both target mutation subsets.

As can be seen in FIG. 4C, the pool of all cancer-observed mutations that are also present in ClinVar is made up of only 5% pathogenic or likely pathogenic mutations while approximately 64% are benign or likely benign. When looking at the pool of mis-splicing mutations one can see that there is a shift in these ratios to where just under 50% of all strictly mis-splicing mutations have evidence of pathogenicity while 11% of these mutations are benign (permutation p value: <0.001). When observing the deleterious mis-splicing mutation intersection one can see this trend becomes even stronger, where 69% of these variants have pathogenic associations and less than 4% are benign (permutation p-value: <0.001). The statistical strength of the latter is relative to the ratios seen in mis-splicing mutations to isolate the effects of Onco-splice scores from SpliceAI's predictions.

Among the diseases associated with the mutations identified among the deleterious mis-splicing variants are several cancer-relevant terms including hereditary cancer predisposition syndrome, familial cancer of breasts, breast-ovarian cancer, ovarian cancer, colorectal cancer, and hepatocellular carcinoma.

### Example 3: Onco-Splice Outperforms Alternative Splicing-Related Pathogenicity Predictors and is Unconstrained by Variant Classification

Many splicing-related pathogenicity predictors have been published. These tools typically leverage machine learning strategies, train classifiers based on a priori knowledge of pathogenicity, and are often constrained to specific mutation types (for example, synonymous SNVs) and regions (for example, intronic). A tabular description of these tools is provided in FIG. 5A. The results from Onco-splice as an end-to-end pathogenicity predictor are compared to results obtained from RegSNPs-Splicing, Reg-SNPs-Intron, S-CAP, TraP, MMSplice, and IntSplice2. A comparison is also made against CADD even though it is not a splicing-specific model and it uses hundreds of other features relating to motifs, conservation estimates, data relating to evolutionary mechanisms, as well as SpliceAI and MMSplice. CADD is orthogonal to Onco-splice and well-established, which allows for an insightful though uneven comparison. 300K mutations obtained from ClinVar using Onco-splice were scored. Pre-computed sets of mutations from all competing models were also scored or obtained. When needed, pathogenicity thresholds were set either using default values provided with each tool's literature or the score marking the top 10% of processed mutations.

FIG. 5B shows the ClinVar labels (pathogenic, benign, or ambiguous) ratio for each of the tool's predicted deleterious mutations. No other tool reaches a ratio of pathogenic to benign mutations as high as is obtained with Onco-splice. To see if more optimal thresholds could define more concentrated sets of pathogenic mutations for each tool, positive predictive values for each tool were obtained based on top-scoring percentiles. As seen in FIG. 5E, only MMSplice, TraP, and CADD obtain PPVs as high as Onco-splice. The performance of all tools were also compare using ROCs in FIG. 5C. Onco-splice's performance approaches that of CADD, which is the only tool analyzed that is non-specific to splicing and that predicts pathogenicity indiscriminately of mechanism; it is a state-of-the-art tool in pathogenicity prediction. All the tools against which Onco-splice was benchmarked have limitations in terms of the range of variant types they can address. Meanwhile, Onco-splice is unconstrained in this regard. Here one can see that even when analyzing each predictor using only the mutations each tool is designed to address, in terms of overall performance, Onco-splice offers the best splicing-related pathogenicity predictions.

Because a training scheme is not used in constructing Onco-splice, it can also be guaranteed that its performance is not affected by data circularity that may affect its ML-utilizing competitors. Additionally, Onco-splice provides insight into mis-splicing mutations that are ORF-bound and non-synonymous, which no other model can handle. These mutations may have distracting and direct effects on the amino acid composition but may have secondary effects on splicing. Similarly, recent investigations point to UTR variants' role in mis-splicing. Several of the mutations Onco-splice identifies as deleterious reside in the 5′UTR region, and these predictions can be used to study their effects further. Ultimately, Onco-splice performs competitively in every regard in the task of pathogenicity prediction without the central reliance on ML as a score generator, without prior knowledge of pathogenicity, without need for a training or optimization scheme, and without variant constraints, all as a secondary task to proteome estimation. Interestingly, the model's scores are not highly correlated to many of its competitors, as shown in FIG. 5D, which indicates that they each may capture different information.

One fundamental aspect of this study emphasizes the importance of silent mutations. To isolate variants that cause changes to the protein exclusively through aberrant slicing, one can define strictly apparently silent mutations as the class of variants that cause predicted splicing aberrations and that do not cause nonsynonymous changes to proteins. When observing strictly apparently silent mutations, the general trends observed in terms of depletion of null occurring mutations, agreement with clinically verified pathogenicity, and correlation between predicted detriment and variant recurrence persist.

### Example 4: Genes Overrepresented with Deleterious Mutations are Enriched with Known Cancer Drivers and Reveal Novel Biomarkers that Improve Patient Survival Estimates

There are several published lists of classical cancer drivers. These lists are often based on non-silent mutations, can be developed either through computational or experimental investigations and ultimately enable targeting for treatment development. If Onco-splice functions properly, it can be reasoned that many of those genes overrepresented with deleterious mis-splicing mutation are known cancer drivers due to direct selection within a cancer cohort. To this end, a search for deleterious mutation-overrepresented genes was carried out using hypergeometric enrichment.

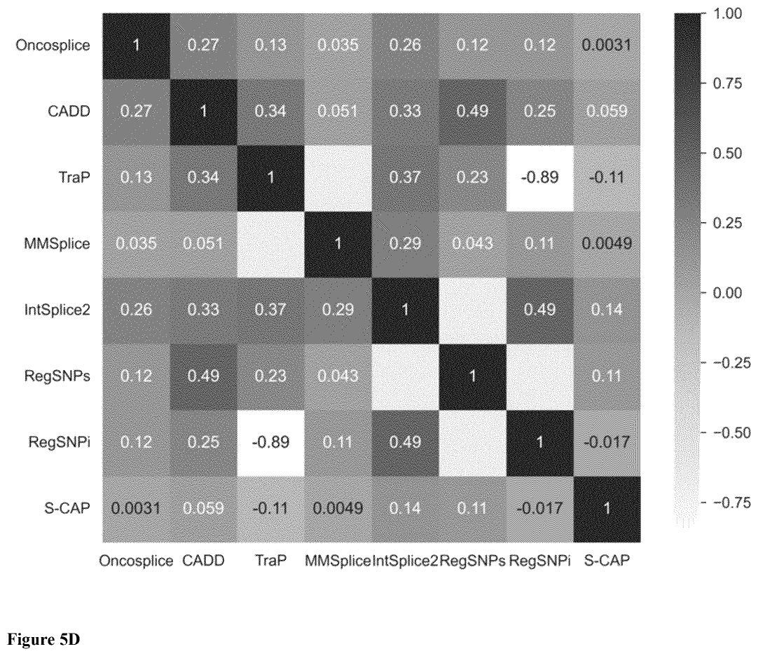

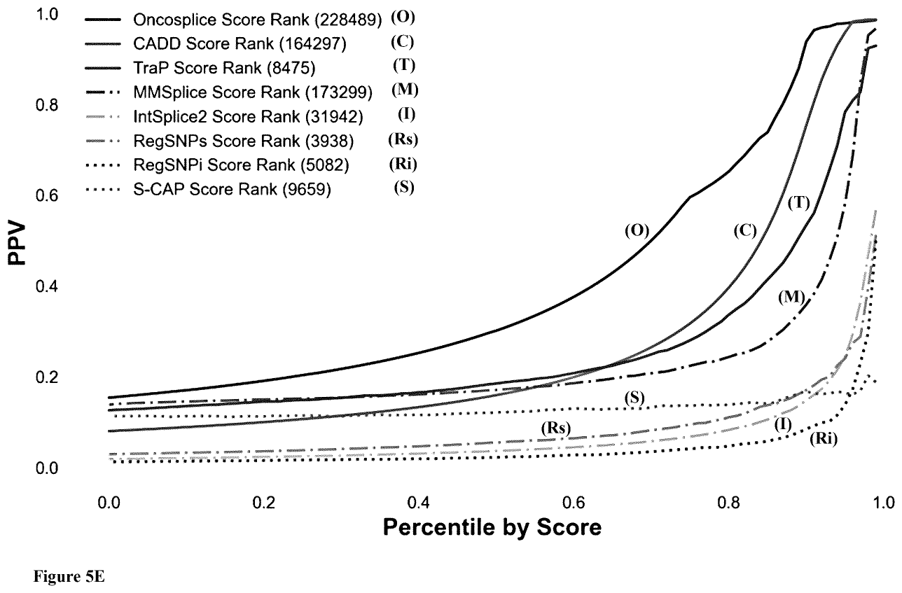

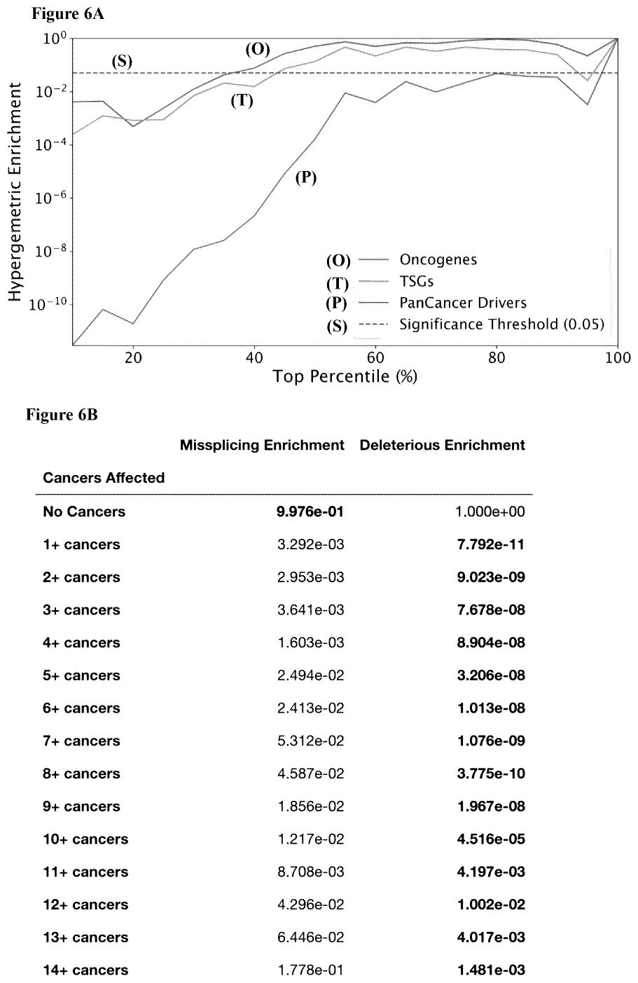

To identify significant genes while controlling for selection bias related to total mutation volume, genes were grouped into 5 distinct bins within each of which selected genes and background genes have insignificantly different mutation volumes, and then genes in each bin are ranked by the ratio of deleterious mis-splicing mutations to all unique mutations. One then scans through the top percentiles across all bins and assesses the identification of drivers. More details on this procedure are available in the Materials and Methods. As can be seen in FIG. 6A, there is strong enrichment of pan-cancer driver genes which reportedly play underlying roles in multiple pathologies. A test for the enrichment of known TSGs and oncogenes is also separately performed using the same procedure and role-specific gene sets. It is seen in FIG. 6A that TSGs are enriched more strongly than oncogenes, indicating either that mis-splicing is a more typical precursor in TSG inactivation than in oncogene modification, or that the scoring strategy implemented better captures behaviors typical of TSG knockout. Quantifying novel protein functionalities that cause an upregulation of activity or change of functionality is a much more difficult task. Enrichment of pan-cancer drivers is also performed in sets of genes that are overrepresented in cancer-specific variant subsets. Moreover, the enrichment of these identified drivers against drivers identified while checking for overrepresentation in the mis-splicing subset is also performed. As can be seen in FIG. 6B, cancer drivers are much more strongly enriched among genes overrepresented by deleterious mutations compared to genes overrepresented by mis-splicing mutations, reinforcing the added value of Onco-splice on top of SpliceAI.

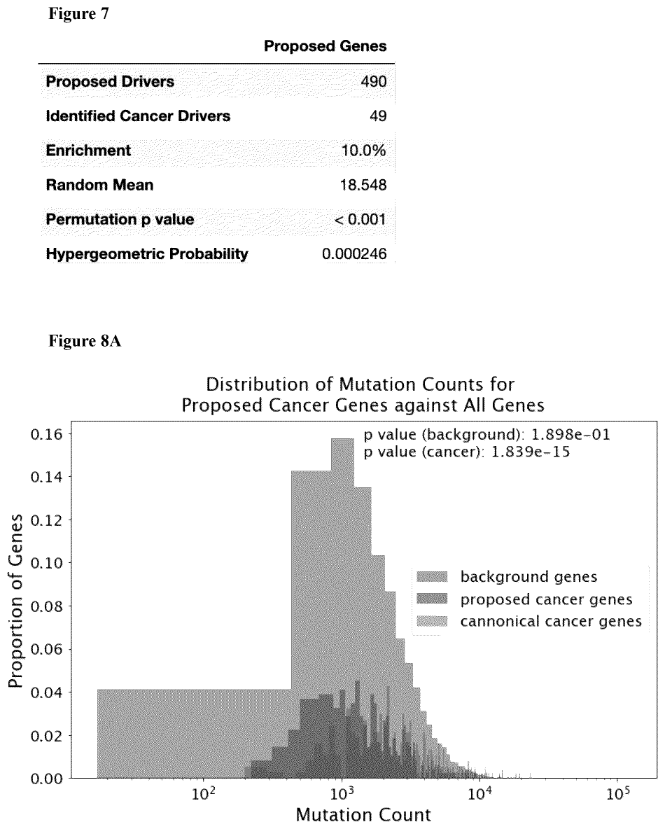

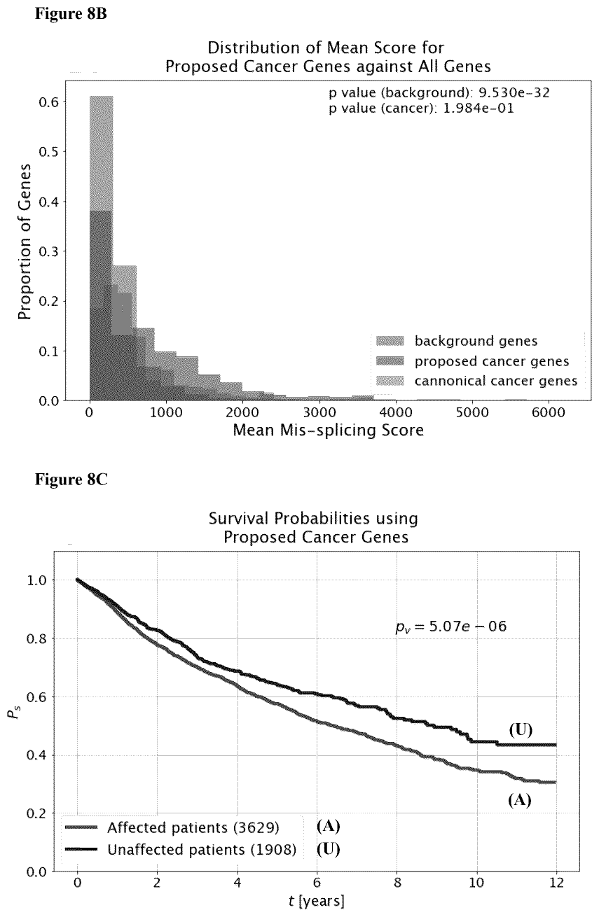

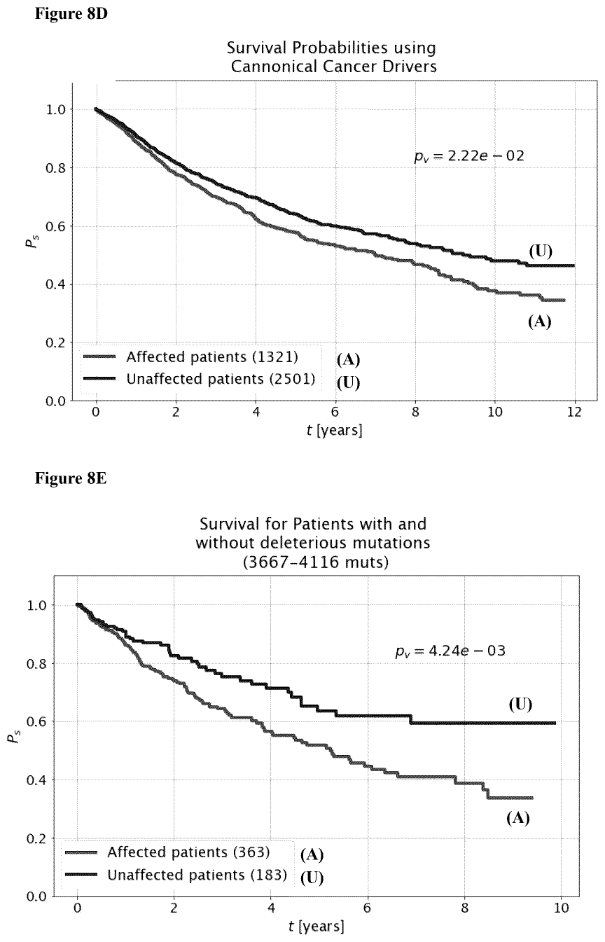

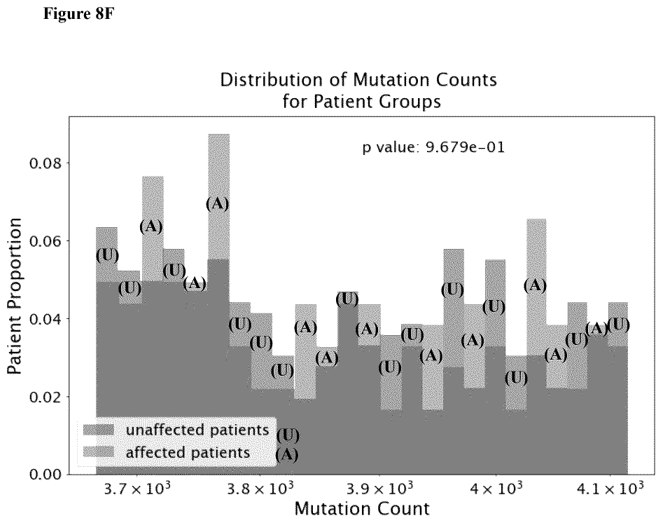

Future cancer treatments and research will be directed toward genes with strong evidence of a potential role in pathogenic mechanisms. Since it has been shown that Onco-splice can capture the enrichment of mutations within canonical cancer drivers and TSGs, one can also use this approach to suggest novel cancer genes by looking at those with the highest enrichment of deleterious mis-splicing events. Therefore, a novel set of potential cancer drivers is suggested. This list includes 490 terms (Table 1) included in the top 5% of overrepresented genes among deleterious mis-splicing mutations. Out of these proposed genes, 49 are canonical pan-cancer drivers. FIG. 7 provides the enrichment of the proposed genes. In essence, these genes can be considered vulnerable to damaging forms of mis-splicing events and to have a role in cancer mechanisms. As seen in FIG. 8A, the proposed cancer drivers come from the same distribution of all genes in terms of the number of mutations they contain, ensuring selection was not dependent on trivial factors. Many relevant cancer-related molecular functions defined by gene ontology gene sets are strongly enriched within this gene set including GTPase activity (adjusted hypergeometric p-value: 6.6E-13), G-protein activity (adjusted hypergeometric p-value: 7.4E-6), and helicase activity (adjusted hypergeometric p-value: 1.9E-3).

To understand the immediate clinical utility of Onco-splice predictions and the proposed cancer drivers, survival estimates were analyzed by identifying patients with deleterious mutations across any of 375 known cancer genes against patients without mis-splicing mutations in those same cancer genes. Similar trials were run where the known cancer genes were replaced with equally sized sets of genes pulled from the novel 490 proposed genes (Table 1). As can be seen in FIGS. 8C-D the segmentation of Kaplan Meier survival estimates for patients using the modified gene list is significantly stronger. This indicates that the novel genes provide immediate clinical prognostic value. Moreover, trials were conducted to control for the mutation volume across patients by segmenting cases into two groups: those with at least one gene affected by a deleterious mutation and those with no genes affected by deleterious mutations. The survival probabilities were then compared for groups of patients such that there is no significant difference between the mutation volume distributions for the affected and unaffected patients in the subset. In many instances, there was no meaningful difference in survival, though when a significant difference was observed it was the patients afflicted by deleterious mutations that had more pessimistic outcomes. FIG. 8E shows the survival probabilities for 546 patients with between 3,667 and 4,116 total mutations. Patients with deleterious mutations have significantly worse survival odds than those without. Moreover, FIG. 8F shows that the patient groups do not have significantly different mutation volumes and that the segmentation is not reliant on trivial factors. In general, data related to survival is troublesome to work with due to missing values and worsening longitudinal record consistency. Regardless, these results indicate that Onco-splice identifies mutations with relation to patient outcome.

TABLE 1
Newly discovered cancer driver genes and their Entrez Gene accession numbers.

| | | Entrez |
|---|---|---|
| Gene | Full name | Gene ID |
| AAAS | aladin WD repeat nucleoporin | 8086 |
| AASDH | aminoadipate-semialdehyde dehydrogenase | 132949 |
| AASS | aminoadipate-semialdehyde synthase | 10157 |
| ABCA12 | ATP binding cassette subfamily A member 12 | 26154 |
| ABCA2 | ATP binding cassette subfamily A member 2 | 20 |
| ABCA8 | ATP binding cassette subfamily A member 8 | 10351 |
| ABHD1 | abhydrolase domain containing 1 | 84696 |
| ADAM8 | ADAM metallopeptidase domain 8 | 101 |
| ADAMTS20 | ADAM metallopeptidase with thrombospondin type 1 motif 20 | 80070 |
| ADAMTSL4 | ADAMTS like 4 | 54507 |
| ADGRV1 | adhesion G protein-coupled receptor V1 | 84059 |
| ADNP | activity dependent neuroprotector homeobox | 23394 |
| AGBL5 | AGBL carboxypeptidase 5 | 60509 |
| AGTPBP1 | ATP/GTP binding carboxypeptidase 1 | 23287 |
| AHCTF1 | AT-hook containing transcription factor 1 | 25909 |
| AK9 | adenylate kinase 9 | 221264 |
| AKAP12 | A-kinase anchoring protein 12 | 9590 |
| AKAP3 | A-kinase anchoring protein 3 | 10566 |
| ANKHD1 | ankyrin repeat and KH domain containing 1 | 54882 |
| ANKRD12 | ankyrin repeat domain 12 | 23253 |
| ANKRD17 | ankyrin repeat domain 17 | 26057 |
| ANKRD31 | ankyrin repeat domain 31 | 256006 |
| ANKRD36C | ankyrin repeat domain 36C | 400986 |
| ANKRD50 | ankyrin repeat domain containing 50 | 57182 |
| APC | APC regulator of WNT signaling pathway | 324 |
| APLP2 | amyloid beta precursor like protein 2 | 334 |
| APOB | apolipoprotein B | 338 |
| ARHGAP23 | Rho GTPase activating protein 23 | 57636 |
| ARHGAP29 | Rho GTPase activating protein 29 | 9411 |
| ARHGAP30 | Rho GTPase activating protein 30 | 257106 |
| ARHGAP32 | Rho GTPase activating protein 32 | 9743 |
| ARHGEF38 | Rho guanine nucleotide exchange factor 38 | 54848 |
| ARID2 | AT-rich interaction domain 2 | 196528 |
| ARID5B | AT-rich interaction domain 5B 17362 | 84159 |
| ARMC5 | armadillo repeat containing 5 | 79798 |
| ASPM | assembly factor for spindle microtubules | 259266 |
| ATG2A | autophagy related 2A | 23130 |
| ATM | ATM serine/threonine kinase | 472 |
| ATOSA | atos homolog A | 56204 |
| ATR | ATR serine/threonine kinase | 545 |
| BAZ1B | bromodomain adjacent to zinc finger domain 1B | 9031 |
| BAZ2A | bromodomain adjacent to zinc finger domain 2A | 11176 |
| BLM | BLM RecQ like helicase | 641 |
| BLTP2 | bridge-like lipid transfer protein family member 2 | 9703 |
| BLTP3B | bridge-like lipid transfer protein family member 3B | 23074 |
| BOC | BOC cell adhesion associated, oncogene regulated | 91653 |
| BRWD1 | bromodomain and WD repeat domain containing 1 | 54014 |
| BTBD8 | BTB domain containing 8 | 284697 |
| C15orf39 | chromosome 15 open reading frame 39 | 56905 |
| CAD | carbamoyl-phosphate synthetase 2, aspartate transcarbamylase, | 790 |
| | and dihydroorotase | |
| CCAR2 | cell cycle and apoptosis regulator 2 | 57805 |
| CCDC136 | coiled-coil domain containing 136 | 64753 |
| CCDC66 | coiled-coil domain containing 66 | 285331 |
| CCDC88A | coiled-coil domain containing 88A | 55704 |
| CCDC88B | coiled-coil domain containing 88B | 283234 |
| CCP110 | centriolar coiled-coil protein 110 | 9738 |
| CCPG1 | cell cycle progression 1 | 9236 |
| CDHR4 | cadherin related family member 4 | 389118 |
| CEP162 | centrosomal protein 162 | 22832 |
| CEP250 | centrosomal protein 250 | 11190 |
| CEP295 | centrosomal protein 295 | 85459 |
| CFAP44 | cilia and flagella associated protein 44 | 55779 |
| CHD6 | chromodomain helicase DNA binding protein 6 | 84181 |
| CHD8 | chromodomain helicase DNA binding protein 8 | 57680 |
| CHD9 | chromodomain helicase DNA binding protein 9 | 80205 |
| CHRD | chordin | 8646 |
| CIZ1 | CDKN1A interacting zinc finger protein 1 | 25792 |
| CLSPN | claspin | 63967 |
| COL12A1 | collagen type XII alpha 1 chain | 1303 |
| CSMD3 | CUB and Sushi multiple domains 3 | 114788 |
| CTNND1 | catenin delta 1 | 1500 |
| DCAF6 | DDB1 and CUL4 associated factor 6 | 55827 |
| DCTN1 | dynactin subunit 1 | 1639 |
| DDIAS | DNA damage induced apoptosis suppressor | 220042 |
| DHX8 | DEAH-box helicase 8 | 1659 |
| DICER1 | dicer 1, ribonuclease III | 23405 |
| DIS3L | DIS3 like exosome 3′-5′ exoribonuclease | 115752 |
| DMXL2 | Dmx like 2 | 23312 |
| DNA2 | DNA replication helicase/nuclease 2 | 1763 |
| DNAH10 | dynein axonemal heavy chain 10 2941 | 196385 |
| DNAH12 | dynein axonemal heavy chain 12 | 201625 |
| DNAH14 | dynein axonemal heavy chain 14 | 127602 |
| DNAH2 | dynein axonemal heavy chain 2 | 146754 |
| DNAH7 | dynein axonemal heavy chain 7 | 56171 |
| DNAH8 | dynein axonemal heavy chain 8 | 1769 |
| DNAH9 | dynein axonemal heavy chain 9 | 1770 |
| DOCK5 | dedicator of cytokinesis 5 | 80005 |
| DTHD1 | death domain containing 1 | 401124 |
| DVL3 | dishevelled segment polarity protein 3 | 1857 |
| DYNC2H1 | dynein cytoplasmic 2 heavy chain 1 | 79659 |
| EDRF1 | erythroid differentiation regulatory factor 1 | 26098 |
| EIF3A | eukaryotic translation initiation factor 3 subunit A | 8661 |
| EIF4ENIF1 | eukaryotic translation initiation factor 4E nuclear import factor 1 | 56478 |
| EPS8L2 | EPS8 like 2 | 64787 |
| ETAA1 | ETAA1 activator of ATR kinase | 54465 |
| EXPH5 | exophilin 5 | 23086 |
| FAM135A | family with sequence similarity 135 member A | 57579 |
| FANCM | FA complementation group M | 57697 |
| FBF1 | Fas binding factor 1 | 85302 |
| FBXL5 | F-box and leucine rich repeat protein 5 | 26234 |
| FBXO11 | F-box protein 11 | 80204 |
| FBXO38 | F-box protein 38 | 81545 |
| FER1L5 | fer-1 like family member 5 | 90342 |
| FILIP1 | filamin A interacting protein 1 | 27145 |
| FOXM1 | forkhead box M1 | 2305 |
| FRMPD1 | FERM and PDZ domain containing 1 | 22844 |
| FRY | FRY microtubule binding protein | 10129 |
| GFM2 | GTP dependent ribosome recycling factor mitochondrial 2 | 84340 |
| GLI1 | GLI family zinc finger 1 | 2735 |
| GNPTAB | N-acetylglucosamine-1-phosphate transferase subunits alpha and | 79158 |
| | beta | |
| GTF2I | general transcription factor IIi | 2969 |
| GTF2IRD2 | GTF2I repeat domain containing 2 | 84163 |
| HECTD1 | HECT domain E3 ubiquitin protein ligase 1 | 25831 |
| HECTD4 | HECT domain E3 ubiquitin protein ligase 4 | 283450 |
| HIF1A | hypoxia inducible factor 1 subunit alpha | 3091 |
| HLTF | helicase like transcription factor | 6596 |
| HMGCR | 3-hydroxy-3-methylglutaryl-CoA reductase | 3156 |
| IBTK | inhibitor of Bruton tyrosine kinase | 25998 |
| ICE2 | interactor of little elongation complex ELL subunit 2 | 79664 |
| IL17RC | interleukin 17 receptor C | 84818 |
| IL6ST | interleukin 6 cytokine family signal transducer | 3572 |
| INPP5F | inositol polyphosphate-5-phosphatase F | 22876 |
| INPPL1 | inositol polyphosphate phosphatase like 1 | 3636 |
| IPO4 | importin 4 19426 | 79711 |
| KAT6A | lysine acetyltransferase 6A | 7994 |
| KCNH2 | potassium voltage-gated channel subfamily H member 2 | 3757 |
| KIAA0232 | KIAA0232 | 9778 |
| KIAA0586 | KIAA0586 | 9786 |
| KIAA0825 | KIAA0825 | 285600 |
| KIAA2026 | KIAA2026 | 158358 |
| KIF23 | kinesin family member 23 | 9493 |
| KIF27 | kinesin family member 27 | 55582 |
| LAMA3 | laminin subunit alpha 3 | 3909 |
| LAMB2 | laminin subunit beta 2 | 3913 |
| LARP1B | La ribonucleoprotein 1B | 55132 |
| LCOR | ligand dependent nuclear receptor corepressor | 84458 |
| LCORL | ligand dependent nuclear receptor corepressor like | 254251 |
| LMTK3 | lemur tyrosine kinase 3 | 114783 |
| LOXHD1 | lipoxygenase homology PLAT domains 1 | 125336 |
| LRIF1 | ligand dependent nuclear receptor interacting factor 1 | 55791 |
| LRP1 | LDL receptor related protein 1 | 4035 |
| LRP2 | LDL receptor related protein 2 | 4036 |
| LRRC9 | leucine rich repeat containing 9 | 341883 |
| LRRK2 | leucine rich repeat kinase 2 | 120892 |
| LTN1 | listerin E3 ubiquitin protein ligase 1 | 26046 |
| MAN2C1 | mannosidase alpha class 2C member 1 | 4123 |
| MAP3K19 | mitogen-activated protein kinase kinase kinase 19 | 80122 |
| MAP4K4 | mitogen-activated protein kinase kinase kinase kinase 4 | 9448 |
| MASTL | microtubule associated serine/threonine kinase like | 84930 |
| MCM7 | minichromosome maintenance complex component 7 | 4176 |
| MCM9 | minichromosome maintenance 9 homologous recombination repair | 254394 |
| | factor | |
| MDN1 | midasin AAA ATPase 1 | 23195 |
| MED1 | mediator complex subunit 1 | 5469 |
| MMRN1 | multimerin 1 | 22915 |
| MPDZ | multiple PDZ domain crumbs cell polarity complex component | 8777 |
| MPHOSPH9 | M-phase phosphoprotein 9 | 10198 |
| MSH2 | mutS homolog 2 | 4436 |
| MTMR4 | myotubularin related protein 4 | 9110 |
| MTOR | mechanistic target of rapamycin kinase | 2475 |
| MYH13 | myosin heavy chain 13 | 8735 |
| MYH2 | myosin heavy chain 2 | 4620 |
| MYO15A | myosin XVA | 51168 |
| MYO9A | myosin IXA | 4649 |
| NCKIPSD | NCK interacting protein with SH3 domain | 51517 |
| NCOR1 | nuclear receptor corepressor 1 | 9611 |
| NF1 | neurofibromin 1 | 4763 |
| NIPBL | NIPBL cohesin loading factor | 25836 |
| NLRX1 | NLR family member X1 | 79671 |
| NOMO3 | NODAL modulator 3 | 408050 |
| NPIPB4 | nuclear pore complex interacting protein family member B4 | 440345 |
| NR3C1 | nuclear receptor subfamily 3 group C member 1 | 2908 |
| NYAP1 | neuronal tyrosine phosphorylated phosphoinositide-3-kinase | 222950 |
| | adaptor 1 | |
| ORC1 | origin recognition complex subunit 1 | 4998 |
| PBRM1 | polybromo 1 | 55193 |
| PCDH1 | protocadherin 1 | 5097 |
| PDZD7 | PDZ domain containing 7 | 79955 |
| PELP1 | proline, glutamate and leucine rich protein 1 | 27043 |
| PER3 | period circadian regulator 3 | 8863 |
| PHF12 | PHD finger protein 12 | 57649 |
| PHF3 | PHD finger protein 3 | 23469 |
| PHLDB1 | pleckstrin homology like domain family B member 1 | 23187 |
| PHRF1 | PHD and ring finger domains 1 | 57661 |
| PIEZO1 | piezo type mechanosensitive ion channel component 1 | 9780 |
| PITPNM1 | phosphatidylinositol transfer protein membrane associated 1 | 9600 |
| PKHD1 | PKHD1 ciliary IPT domain containing fibrocystin/polyductin | 5314 |
| PLA2G2C | phospholipase A2 group IIC | 391013 |
| PLA2G2D | phospholipase A2 group IID | 26279 |
| PLAA | phospholipase A2 activating protein | 9373 |
| PLAC8 | placenta associated 8 | 51316 |
| PLAC9 | placenta associated 9 | 219348 |
| PLCG1 | phospholipase C gamma 1 | 5335 |
| PLEKHF1 | pleckstrin homology and FYVE domain containing 1 | 79156 |
| PLEKHF2 | pleckstrin homology and FYVE domain containing 2 | 79666 |
| PLEKHJ1 | pleckstrin homology domain containing J1 | 55111 |
| PLIN5 | perilipin 5 | 440503 |
| PLLP | plasmolipin | 51090 |
| PMP2 | peripheral myelin protein 2 | 5375 |
| PMP22 | peripheral myelin protein 22 | 5376 |
| PMS1 | PMS1 homolog 1, mismatch repair system component | 5378 |
| PNMT | phenylethanolamine N-methyltransferase | 5409 |
| PNOC | prepronociceptin | 5368 |
| PNPO | pyridoxamine 5′-phosphate oxidase | 55163 |
| PNRC1 | proline rich nuclear receptor coactivator 1 | 10957 |
| POLE3 | DNA polymerase epsilon 3, accessory subunit | 54107 |
| POLK | DNA polymerase kappa | 51426 |
| POLR1D | RNA polymerase I and III subunit D | 51082 |
| POLR2F | RNA polymerase II, I and III subunit F | 5435 |
| POLR2H | RNA polymerase II, I and III subunit H | 5437 |
| POLR2J2 | RNA polymerase II subunit J2 | 246721 |
| POLR2K | RNA polymerase II, I and III subunit K | 5440 |
| POMC | proopiomelanocortin | 5443 |
| POP5 | POP5 homolog, ribonuclease P/MRP subunit | 51367 |
| POU1F1 | POU class 1 homeobox 1 | 5449 |
| PPCDC | phosphopantothenoylcysteine decarboxylase | 60490 |
| PPCS | phosphopantothenoylcysteine synthetase | 79717 |
| PPDPF | pancreatic progenitor cell differentiation and proliferation factor | 79144 |
| PPIG | peptidylprolyl isomerase G | 9360 |
| PPIL3 | peptidylprolyl isomerase like 3 | 53938 |
| PPM1M | protein phosphatase, Mg2+/Mn2+ dependent 1M | 132160 |
| PPM1N | protein phosphatase, Mg2+/Mn2+ dependent 1N (putative) | 147699 |
| PPP1R11 | protein phosphatase 1 regulatory inhibitor subunit 11 | 6992 |
| PPP6R1 | protein phosphatase 6 regulatory subunit 1 | 22870 |
| PRDM1 | PR/SET domain 1 | 639 |
| PRDM11 | PR/SET domain 11 | 56981 |
| PRICKLE1 | prickle planar cell polarity protein 1 | 144165 |
| PRPF40B | pre-mRNA processing factor 40 homolog B | 25766 |
| PRR30 | proline rich 30 | 339779 |
| PRR4 | proline rich 4 | 5554 |
| PRRT1 | proline rich transmembrane protein 1 | 80863 |
| PRRT2 | proline rich transmembrane protein 2 | 112476 |
| PRRT3 | proline rich transmembrane protein 3 | 285368 |
| PRRT4 | proline rich transmembrane protein 4 | 401399 |
| PRSS21 | serine protease 21 | 10942 |
| PRSS22 | serine protease 22 | 64063 |
| PRSS8 | serine protease 8 | 5652 |
| PRTN3 | proteinase 3 | 5657 |
| PSENEN | presenilin enhancer, gamma-secretase subunit | 55851 |
| PSKH1 | protein serine kinase H1 | 5681 |
| PSMA7 | proteasome 20S subunit alpha 7 | 5688 |
| PSMB5 | proteasome 20S subunit beta 5 | 5693 |
| PSMB6 | proteasome 20S subunit beta 6 | 5694 |
| PSMC3IP | PSMC3 interacting protein | 29893 |
| PSMD8 | proteasome 26S subunit, non-ATPase 8 | 5714 |
| PSMD9 | proteasome 26S subunit, non-ATPase 9 | 5715 |
| PSME1 | proteasome activator subunit 1 | 5720 |
| PSME2 | proteasome activator subunit 2 | 5721 |
| PSMG3 | proteasome assembly chaperone 3 | 84262 |
| PSMG4 | proteasome assembly chaperone 4 | 389362 |
| PSRC1 | proline and serine rich coiled-coil 1 | 84722 |
| PTAR1 | protein prenyltransferase alpha subunit repeat containing 1 | 375743 |
| PTCRA | pre T cell antigen receptor alpha | 171558 |
| PTGDR | prostaglandin D2 receptor | 5729 |
| PTGER2 | prostaglandin E receptor 2 | 5732 |
| PTGIR | prostaglandin I2 receptor | 5739 |
| PTH | parathyroid hormone | 5741 |
| PTHLH | parathyroid hormone like hormone | 5744 |
| PTP4A1 | protein tyrosine phosphatase 4A1 | 7803 |
| PTP4A2 | protein tyrosine phosphatase 4A2 | 8073 |
| PTP4A3 | protein tyrosine phosphatase 4A3 | 11156 |
| PTPMT1 | protein tyrosine phosphatase mitochondrial 1 | 114971 |
| PTRH1 | peptidyl-tRNA hydrolase 1 homolog | 138428 |
| PTS | 6-pyruvoyltetrahydropterin synthase | 5805 |
| PUS1 | pseudouridine synthase 1 | 80324 |
| PUS3 | pseudouridine synthase 3 | 83480 |
| PWWP2A | PWWP domain containing 2A | 114825 |
| PXMP2 | peroxisomal membrane protein 2 | 5827 |
| PXN | paxillin | 5829 |
| PYCARD | PYD and CARD domain containing | 29108 |
| PYCR1 | pyrroline-5-carboxylate reductase 1 | 5831 |
| PYCR2 | pyrroline-5-carboxylate reductase 2 | 29920 |
| PYGO2 | pygopus family PHD finger 2 | 90780 |
| QPRT | quinolinate phosphoribosyltransferase | 23475 |
| R3HDM1 | R3H domain containing 1 | 23518 |
| R3HDM4 | R3H domain containing 4 | 91300 |
| RAB11A | RAB11A, member RAS oncogene family | 8766 |
| RAB11B | RAB11B, member RAS oncogene family | 9230 |
| RAB11FIP2 | RAB11 family interacting protein 2 | 22841 |
| RAB1A | RAB1A, member RAS oncogene family | 5861 |
| RAB1B | RAB1B, member RAS oncogene family | 81876 |
| RAB23 | RAB23, member RAS oncogene family | 51715 |
| RAB24 | RAB24, member RAS oncogene family | 53917 |
| RAB26 | RAB26, member RAS oncogene family | 25837 |
| RAB29 | RAB29, member RAS oncogene family | 8934 |
| RAB2B | RAB2B, member RAS oncogene family | 84932 |
| RAB30 | RAB30, member RAS oncogene family | 27314 |
| RAB33B | RAB33B, member RAS oncogene family | 83452 |
| RAB34 | RAB34, member RAS oncogene family | 83871 |
| RAB35 | RAB35, member RAS oncogene family | 11021 |
| RAB3A | RAB3A, member RAS oncogene family | 5864 |
| RAB3D | RAB3D, member RAS oncogene family | 9545 |
| RAB40B | RAB40B, member RAS oncogene family | 10966 |
| RAB40C | RAB40C, member RAS oncogene family | 57799 |
| RAB4A | RAB4A, member RAS oncogene family | 5867 |
| RAB4B | RAB4B, member RAS oncogene family | 53916 |
| RAB5A | RAB5A, member RAS oncogene family | 5868 |
| RAB5B | RAB5B, member RAS oncogene family | 5869 |
| RAB5C | RAB5C, member RAS oncogene family | 5878 |
| RAB8A | RAB8A, member RAS oncogene family | 4218 |
| RABL2A | RAB, member of RAS oncogene family like 2A | 11159 |
| RAC1 | Rac family small GTPase 1 | 5879 |
| RAC2 | Rac family small GTPase 2 | 5880 |
| RAD1 | RAD1 checkpoint DNA exonuclease | 5810 |
| RAD51 | RAD51 recombinase | 5888 |
| RAD9B | RAD9 checkpoint clamp component B | 144715 |
| RAET1E | retinoic acid early transcript 1E | 135250 |
| RALB | RAS like proto-oncogene B | 5899 |
| RALGAPA1 | Ral GTPase activating protein catalytic subunit alpha 1 | 253959 |
| RALY | RALY heterogeneous nuclear ribonucleoprotein | 22913 |
| RAMP3 | receptor activity modifying protein 3 | 10268 |
| RANBP6 | RAN binding protein 6 | 26953 |
| RAPH1 | Ras association (RalGDS/AF-6) and pleckstrin homology domains 1 | 65059 |
| | 1443 | |
| RARRES1 | retinoic acid receptor responder 1 9867 | 5918 |
| RASGRP2 | RAS guanyl releasing protein 2 9879 | 10235 |
| RASGRP4 | RAS guanyl releasing protein 4 | 115727 |
| RASSF3 | Ras association domain family member 3 14271 | 283349 |
| RASSF5 | Ras association domain family member 5 | 83593 |
| RASSF6 | Ras association domain family member 6 | 166824 |
| RASSF8 | Ras association domain family member 8 | 11228 |
| RAVER1 | ribonucleoprotein, PTB binding 1 | 125950 |
| RBAK | RB associated KRAB zinc finger | 57786 |
| RBCK1 | RANBP2-type and C3HC4-type zinc finger containing 1 | 10616 |
| RBFA | ribosome binding factor A | 79863 |
| RBM12 | RNA binding motif protein 12 | 10137 |
| RBM14 | RNA binding motif protein 14 | 10432 |
| RBM15 | RNA binding motif protein 15 | 64783 |
| RBM17 | RNA binding motif protein 17 | 84991 |
| RBM22 | RNA binding motif protein 22 | 55696 |
| RBM42 | RNA binding motif protein 42 | 79171 |
| RBM43 | RNA binding motif protein 43 | 375287 |
| RBM45 | RNA binding motif protein 45 | 129831 |
| RBM47 | RNA binding motif protein 47 | 54502 |
| RBSN | rabenosyn, RAB effector | 64145 |
| RCBTB1 | RCC1 and BTB domain containing protein 1 | 55213 |
| RCBTB2 | RCC1 and BTB domain containing protein 2 | 1102 |
| RCC1 | regulator of chromosome condensation 1 | 1104 |
| RCC2 | regulator of chromosome condensation 2 | 55920 |
| RCSD1 | RCSD domain containing 1 | 92241 |
| RDH12 | retinol dehydrogenase 12 | 145226 |
| RDM1 | RAD52 motif containing 1 | 201299 |
| REG4 | regenerating family member 4 | 83998 |
| RELB | RELB proto-oncogene, NF-kB subunit | 5971 |
| RELN | reelin | 5649 |
| RERGL | RERG like | 79785 |
| REST | RE1 silencing transcription factor | 5978 |
| RFC3 | replication factor C subunit 3 | 5983 |
| RFT1 | RFT1 homolog | 91869 |
| RFX5 | regulatory factor X5 | 5993 |
| RFX8 | regulatory factor X8 | 731220 |
| RGMA | repulsive guidance molecule BMP co-receptor a | 56963 |
| RGMB | repulsive guidance molecule BMP co-receptor b | 285704 |
| RGPD8 | RANBP2 like and GRIP domain containing 8 | 727851 |
| RGR | retinal G protein coupled receptor | 5995 |
| RGS17 | regulator of G protein signaling 17 | 26575 |
| RGS20 | regulator of G protein signaling 20 | 8601 |
| RGS4 | regulator of G protein signaling 4 | 5999 |
| RGS8 | regulator of G protein signaling 8 | 85397 |
| RHAG | Rh associated glycoprotein | 6005 |
| RHBDD1 | rhomboid domain containing 1 | 84236 |
| RHBDD2 | rhomboid domain containing 2 | 57414 |
| RHBDL2 | rhomboid like 2 | 54933 |
| RHD | Rh blood group D antigen | 6007 |
| RHEB | Ras homolog, mTORC1 binding | 6009 |
| RHOBTB1 | Rho related BTB domain containing 1 | 9886 |
| RHOJ | ras homolog family member J | 57381 |
| RIC3 | RIC3 acetylcholine receptor chaperone | 79608 |
| RIC8A | RIC8 guanine nucleotide exchange factor A | 60626 |
| RIC8B | RIC8 guanine nucleotide exchange factor B | 55188 |
| RILPL1 | Rab interacting lysosomal protein like 1 | 353116 |
| RIMKLB | ribosomal modification protein rimK like family member B | 57494 |
| RIN1 | Ras and Rab interactor 1 | 9610 |
| RMND5B | required for meiotic nuclear division 5 homolog B | 64777 |
| RNASEL | ribonuclease L | 6041 |
| RNASET2 | ribonuclease T2 | 8635 |
| RND3 | Rho family GTPase 3 | 390 |
| RNF114 | ring finger protein 114 | 55905 |
| RNF135 | ring finger protein 135 | 84282 |
| RNF138 | ring finger protein 138 | 51444 |
| RNF14 | ring finger protein 14 | 9604 |
| RNF141 | ring finger protein 141 | 50862 |
| RNF145 | ring finger protein 145 | 153830 |
| RNF182 | ring finger protein 182 | 221687 |
| RNF185 | ring finger protein 185 | 91445 |
| RNF19B | ring finger protein 19B | 127544 |
| RNF2 | ring finger protein 2 | 6045 |
| RNF212B | ring finger protein 212B | 100507650 |
| RNF34 | ring finger protein 34 | 80196 |
| RNF41 | ring finger protein 41 | 10193 |
| RNF6 | ring finger protein 6 | 6049 |
| RNF8 | ring finger protein 8 | 9025 |
| RNH1 | ribonuclease/angiogenin inhibitor 1 | 6050 |
| ROCK2 | Rho associated coiled-coil containing protein kinase 2 | 9475 |
| ROPN1 | rhophilin associated tail protein 1 | 54763 |
| ROPN1B | rhophilin associated tail protein 1B | 152015 |
| RPA2 | replication protein A2 | 6118 |
| RPA3 | replication protein A3 | 6119 |
| RPL12 | ribosomal protein L12 | 6136 |
| RPL14 | ribosomal protein L14 | 9045 |
| RPL18 | ribosomal protein L18 | 6141 |
| RPL27A | ribosomal protein L27a | 6157 |
| RPL37A | ribosomal protein L37a | 6168 |
| RPL4 | ribosomal protein L4 | 6124 |
| RPL5 | ribosomal protein L5 | 6125 |
| RPP14 | ribonuclease P/MRP subunit p14 | 11102 |
| RPP40 | ribonuclease P/MRP subunit p40 | 10799 |
| RPRD1A | regulation of nuclear pre-mRNA domain containing 1A | 55197 |
| RPS17 | ribosomal protein S17 | 6218 |
| RPS21 | ribosomal protein S21 | 6227 |
| RPS24 | ribosomal protein S24 1041 | 6229 |
| RPS3 | ribosomal protein S3 | 6188 |
| RPS3A | ribosomal protein S3A 10421 | 6189 |
| RPS6KA4 | ribosomal protein S6 kinase A4 | 8986 |
| RPUSD2 | RNA pseudouridine synthase domain containing 2 24180 | 27079 |
| RRAS2 | RAS related 2 | 22800 |
| RREB1 | ras responsive element binding protein 1 10449 | 6239 |
| RRM2 | ribonucleotide reductase regulatory subunit M2 10452 | 6241 |
| RRP8 | ribosomal RNA processing 8 | 23378 |
| RSBN1L | round spermatid basic protein 1 like | 222194 |
| RSPH1 | radial spoke head component 1 | 89765 |
| RSPH14 | radial spoke head 14 homolog | 27156 |
| RSPH9 | radial spoke head component 9 | 221421 |
| RYR3 | ryanodine receptor 3 | 6263 |
| SART3 | spliceosome associated factor 3, U4/U6 recycling protein | 9733 |
| SECISBP2L | SECIS binding protein 2 like | 9728 |
| SETD5 | SET domain containing 5 | 55209 |
| SGSM2 | small G protein signaling modulator 2 | 9905 |
| SHPRH | SNF2 histone linker PHD RING helicase | 257218 |
| SIN3B | SIN3 transcription regulator family member B 19354 | 23309 |
| SKIC2 | SKI2 subunit of superkiller complex | 6499 |
| SLC12A4 | solute carrier family 12 member 4 | 6560 |
| SLC12A9 | solute carrier family 12 member 9 | 56996 |
| SMARCAD1 | SWI/SNF-related, matrix-associated actin-dependent regulator of | 56916 |
| | chromatin, subfamily a, containing DEAD/H box 1 1839 | |
| SMG7 | SMG7 nonsense mediated mRNA decay factor 16792 | 9887 |
| SNX13 | sorting nexin 13 | 23161 |
| SNX14 | sorting nexin 14 | 57231 |
| SPEF2 | sperm flagellar 2 26293 | 79925 |
| SPEG | striated muscle enriched protein kinase | 10290 |
| SPG11 | SPG11 vesicle trafficking associated, spatacsin 11226 | 80208 |
| SPTBN1 | spectrin beta, non-erythrocytic 1 | 6711 |
| SRCAP | Snf2 related CREBBP activator protein | 10847 |
| SSH1 | slingshot protein phosphatase 1 | 54434 |
| SVEP1 | sushi, von Willebrand factor type A, EGF and pentraxin domain | 79987 |
| | containing 1 | |
| SYCP2 | synaptonemal complex protein 2 | 10388 |
| SYNE2 | spectrin repeat containing nuclear envelope protein 2 | 23224 |
| SYNJ1 | synaptojanin 1 | 8867 |
| SYNM | synemin | 23336 |
| SYNRG | synergin gamma | 11276 |
| SZT2 | SZT2 subunit of KICSTOR complex | 23334 |
| TDRD12 | tudor domain containing 12 | 91646 |
| TJP1 | tight junction protein 1 | 7082 |
| TLR4 | toll like receptor 4 | 7099 |
| TNS2 | tensin 2 | 23371 |
| TRRAP | transformation/transcription domain associated protein | 8295 |
| TUT1 | terminal uridylyl transferase 1, U6 snRNA-specific | 64852 |
| TYRO3 | TYRO3 protein tyrosine kinase | 7301 |
| UACA | uveal autoantigen with coiled-coil domains and ankyrin repeats | 55075 |
| UBR4 | ubiquitin protein ligase E3 component n-recognin 4 | 23352 |
| UBR5 | ubiquitin protein ligase E3 component n-recognin 5 | 51366 |
| UNC79 | unc-79 homolog, NALCN channel complex subunit | 57578 |
| UNC80 | unc-80 homolog, NALCN channel complex subunit | 285175 |
| USH2A | usherin | 7399 |
| USP33 | ubiquitin specific peptidase 33 | 23032 |
| USPL1 | ubiquitin specific peptidase like 1 | 10208 |
| VCAN | versican | 1462 |
| VILL | villin like | 50853 |
| VPS13C | vacuolar protein sorting 13 homolog C | 54832 |
| VPS13D | vacuolar protein sorting 13 homolog D | 55187 |
| WDR6 | WD repeat domain 6 | 11180 |
| WIZ | WIZ zinc finger | 58525 |
| YTHDC2 | YTH domain containing 2 | 64848 |
| YY1AP1 | YY1 associated protein 1 | 55249 |
| ZBTB20 | zinc finger and BTB domain containing 20 | 26137 |
| ZC3H6 | zinc finger CCCH-type containing 6 | 376940 |
| ZC3H7A | zinc finger CCCH-type containing 7A | 29066 |
| ZCCHC2 | zinc finger CCHC-type containing 2 | 54877 |
| ZFYVE16 | zinc finger FYVE-type containing 16 | 9765 |
| ZHX1 | zinc fingers and homeoboxes 1 | 11244 |
| ZHX3 | zinc fingers and homeoboxes 3 | 23051 |
| ZMYM1 | zinc finger MYM-type containing 1 26253 | 79830 |
| ZMYM6 | zinc finger MYM-type containing 6 | 9204 |
| ZNF208 | zinc finger protein 208 | 7757 |
| ZNF226 | zinc finger protein 226 | 7769 |
| ZNF268 | zinc finger protein 268 | 10795 |
| ZNF280D | zinc finger protein 280D 25953 | 54816 |
| ZNF292 | zinc finger protein 292 | 23036 |
| ZNF616 | zinc finger protein 616 | 90317 |
| ZNF644 | zinc finger protein 644 | 84146 |
| ZNF780B | zinc finger protein 780B | 163131 |
| ZNF814 | zinc finger protein 814 | 730051 |
| ZNF841 | zinc finger protein 841 | 284371 |
| ZSCAN20 | zinc finger and SCAN domain containing 20 | 7579 |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of identifying a deleterious mutation in a cancer, the method comprising:
a. receiving mutation data from said cancer, wherein said mutation data comprises genomic sequence changes as compared to a healthy control genome;
b. selecting from said received mutation data a mutation that disrupts or creates a splice donor or splice acceptor site within a transcribed region;
c. for a selected mutation calculating all possible resultant spliced mRNA transcripts that can be produced from said transcribed region;
d. for all possible resultant spliced mRNA transcripts determining all possible amino acid sequences encoded; and
e. calculate a functional divergence score for said selected mutation based on the determined amino acid sequences as compared to a healthy control sequence, wherein said functional divergence score is a measure of the severity in protein function alteration present in said cancer as compared to a healthy control, and wherein a functional divergence score beyond a predetermined threshold indicates said selected mutation is a deleterious mutation, optionally wherein said predetermined threshold for said functional divergence score is 690;
thereby identifying a deleterious mutation in a cancer.

2. The method of claim 1, wherein said cancer is selected from breast cancer, uterine cancer, head and neck cancer, brain cancer, prostate cancer, lung cancer, thyroid cancer, skin cancer, stomach cancer, bladder cancer, urothelial cancer, colon cancer, liver cancer, ovarian cancer, kidney cancer, cervical cancer, bone cancer, connective tissue cancer, esophageal cancer, pancreatic cancer, adrenal cancer, neuroendocrine cancer, rectal cancer, leukemia, testicular cancer, uveal cancer, bile duct cancer and lymphoma.

3. The method of claim 1, wherein said received mutation data comprises whole exosome sequencing (WES) data from a sample comprising cancer DNA.

4. (canceled)

5. The method of claim 1, wherein at least one of:
a. said healthy control genome is a consensus genome for a species in which said cancer originated or wherein said healthy control genome is a genome in a non-cancerous cell of the same cell type as said cancer;
b. said sample is selected from a tumor sample and a bodily fluid sample, wherein said bodily fluid comprises cancer cells or cell free cancer DNA; and
c. an identified deleterious mutation in a gene indicates said gene is a cancer driver gene in said cancer.

6. The method of claim 1, wherein said received mutation data comprises mutations within exons, introns, and untranslated regions (UTRs).

7. The method of claim 1, wherein a splice donor site comprises the sequence GU and a splice acceptor site comprises the sequence AG, wherein a mutation that disrupts a splice donor or acceptor site is a mutation that disrupts an annotated splice donor or acceptor site in the genome of the species from which said cancer originated or both.

8. The method of claim 1, wherein said selecting a mutation that disrupt or creates a splice donor or splice acceptor site comprises applying a trained machine learning algorithm to a genomic sequence comprising said mutation and wherein said trained machine learning algorithm outputs all predicted splice donor and splice acceptor sites affected by said mutation.

9. The method of claim 8, wherein said trained machine learning algorithm is first applied to said genomic sequence without said mutation and said machine learning algorithm outputs all predicted splice donor and splice acceptor sties in said genomic sequence.

10. The method of claim 9, wherein said machine learning algorithm outputs a probability score for a dinucleotide being a splice donor or splice acceptor site and wherein a site predicted to be affected by said mutation is a site whose score changes by at least a predetermined threshold from a probability score in the genomic sequence without said mutation to a probability score in the genomic sequence with the mutation, optionally wherein said predetermined threshold is 0.5.

11. (canceled)

12. The method of claim 8, wherein said genomic sequence comprises at least 10,000 nucleotides in addition to the mutation, optionally wherein said genomic sequence comprises at least 15,000 nucleotides in addition to the mutation, said genomic sequence comprises at least 5000 nucleotides upstream of said mutation and at least 5000 nucleotides downstream of said mutation, optionally wherein said genomic sequence comprises at least 7500 nucleotides upstream of said mutation and at least 7500 nucleotides downstream of said mutation or both.

13. (canceled)

14. (canceled)

15. The method of claim 1, wherein said calculating all possible resultant spliced mRNA transcripts comprises producing a list of all transcripts that can be created by linking a donor splice site to each downstream acceptor splice site that is present before the next donor splice site, optionally wherein any transcript comprising a non-canonical exon comprising greater than 2000 nucleotides is discarded.

16. (canceled)

17. The method of claim 1, wherein said determining the amino acid sequence encoded comprises determining all possible translation initiation sites (TIS) and from each TIS determining the amino acids encoded until a translation termination site (TTS) is reached.

18. The method of claim 1, wherein said calculating a functional divergence score is based on a per residue evolutionary conservation values, and wherein divergence score is proportional or inversely proportional to the evolutionary conservation value of a residue present in said healthy control sequence and altered by said mutation.

19. The method of claim 18, wherein at least one of:
a. a per residue evolutionary conservation value is calculated by a method comprising producing a multiple sequence alignment (MSA) from sequences of homologous proteins from different species and calculating a conservation value of each residue across the MSA;
b. said calculating a functional divergence score comprises calculating a deletion score comprising the sum of the per residue evolutionary conservation values for all residues not present in the determined amino acid sequence divided by the sum of all per residue evolutionary conservation values of the amino acid sequence, calculating an insertion score comprising the sum of the per residue evolutionary conservation values for all 4 amino acid residue blocks interrupted by an insertion divided by the sum of all per residue evolutionary conservation values of the amino acid sequence and multiplying the deletion score by the insertion score to produce a disruption score; and
c. said functional divergence score is 1-said disruption score and beyond said predetermined threshold is below said predetermined threshold.

20. (canceled)

21. (canceled)

22. (canceled)

23. The method of claim 1, wherein said method comprises calculating a functional divergence score for all mutations that disrupt or create a splice donor or splice acceptor site, optionally wherein said predetermined threshold is a bottom percentile of the mutations that produces the most functional divergence, wherein said percentile is the bottom 21^{st} percentile of mutations by functional divergence score, wherein a lower score indicates greater divergence or both.

24. (canceled)

25. (canceled)

26. The method of claim 1, wherein said calculating a functional divergence score comprises:
a. determining a functional divergence score for all determined amino acid sequences;
b. for each mRNA transcript averaging the functional divergence scores of all possible determined amino acid sequences; and
c. select the averaged functional divergence score indicating the greatest divergence as the functional divergence score for said mutation.

27. (canceled)

28. A method of prognosing a subject suffering from cancer, the method comprising determining deleterious mutations in said cancer by a method comprising a method of claim 1, wherein the number of deleterious mutations present is inversely related to the prognosis of said subject, thereby prognosing a subject suffering from cancer.

29. (canceled)

30. The method of claim 28, wherein at least one of:
a. said number of deleterious mutations is normalized to the total number of mutations in the cancer or the total number of mutations that disrupt or create a splice donor or splice acceptor site;
b. said determining deleterious mutations comprises determining all deleterious mutations; and
c. said determining deleterious mutations comprises excluding mutations identified in control healthy subjects or tissue.

31. A method of evaluating or detecting a cancer or precancerous cell in a subject, the method comprising:
a. receiving a sample from said subject comprising genomic DNA; and
b. identifying in said genomic DNA a mutation that disrupts or creates a splice donor or splice acceptor site within a gene selected from: AAAS, AASDH, AASS, ABCA12, ABCA2, ABCA8, ABHD1, ADAM8, ADAMTS20, ADAMTSL4, ADGRV1, ADNP, AGBL5, AGTPBP1, AHCTF1, AK9, AKAP12, AKAP3, ANKHD1, ANKRD12, ANKRD17, ANKRD31, ANKRD36C, ANKRD50, APC, APLP2, APOB, ARHGAP23, ARHGAP29, ARHGAP30, ARHGAP32, ARHGEF38, ARID2, ARID5B, ARMC5, ASPM, ATG2A, ATM, ATOSA, ATR, BAZIB, BAZ2A, BLM, BLTP2, BLTP3B, BOC, BRWD1, BTBD8, C15orf39, CAD, CCAR2, CCDC136, CCDC66, CCDC88A, CCDC88B, CCP110, CCPG1, CDHR4, CEP162, CEP250, CEP295, CFAP44, CHD6, CHD8, CHD9, CHRD, CIZ1, CLSPN, COL12A1, CSMD3, CTNND1, DCAF6, DCTN1, DDIAS, DHX8, DICER1, DIS3L, DMXL2, DNA2, DNAH10, DNAH12, DNAH14, DNAH2, DNAH7, DNAH8, DNAH9, DOCK5, DTHD1, DVL3, DYNC2H1, EDRF1, EIF3A, EIF4ENIF1, EPS8L2, ETAA1, EXPH5, FAM135A, FANCM, FBF1, FBXL5, FBXO11, FBXO38, FER1L5, FILIP1, FOXM1, FRMPD1, FRY, GFM2, GLI1, GNPTAB, GTF2I, GTF2IRD2, HECTD1, HECTD4, HIF1A, HLTF, HMGCR, IBTK, ICE2, IL17RC, IL6ST, INPP5F, INPPL1, IPO4, KAT6A, KCNH2, KIAA0232, KIAA0586, KIAA0825, KIAA2026, KIF23, KIF27, LAMA3, LAMB2, LARP1B, LCOR, LCORL, LMTK3, LOXHD1, LRIF1, LRP1, LRP2, LRRC9, LRRK2, LTN1, MAN2C1, MAP3K19, MAP4K4, MASTL, MCM7, MCM9, MDN1, MED1, MMRN1, MPDZ, MPHOSPH9, MSH2, MTMR4, MTOR, MYH13, MYH2, MYO15A, MY09A, NCKIPSD, NCOR1, NF1, NIPBL, NLRX1, NOMO3, NPIPB4, NR3C1, NYAP1, ORC1, PBRM1, PCDH1, PDZD7, PELP1, PER3, PHF12, PHF3, PHLDB1, PHRF1, PIEZO1, PITPNM1, PKHD1, PLA2G2C, PLA2G2D, PLAA, PLAC8, PLAC9, PLCG1, PLEKHF1, PLEKHF2, PLEKHJ1, PLIN5, PLLP, PMP2, PMP22, PMS1, PNMT, PNOC, PNPO, PNRC1, POLE3, POLK, POLR1D, POLR2F, POLR2H, POLR2J2, POLR2K, POMC, POP5, POUIF1, PPCDC, PPCS, PPDPF, PPIG, PPIL3, PPM1M, PPM1N, PPP1R11, PPP6R1, PRDM1, PRDM11, PRICKLE1, PRPF40B, PRR30, PRR4, PRRT1, PRRT2, PRRT3, PRRT4, PRSS21, PRSS22, PRSS8, PRTN3, PSENEN, PSKH1, PSMA7, PSMB5, PSMB6, PSMC3IP, PSMD8, PSMD9, PSME1, PSME2, PSMG3, PSMG4, PSRC1, PTAR1, PTCRA, PTGDR, PTGER2, PTGIR, PTH, PTHLH, PTP4A1, PTP4A2, PTP4A3, PTPMT1, PTRH1, PTS, PUS1, PUS3, PWWP2A, PXMP2, PXN, PYCARD, PYCR1, PYCR2, PYGO2, QPRT, R3HDM1, R3HDM4, RAB11A, RAB11B, RAB11FIP2, RAB1A, RAB1B, RAB23, RAB24, RAB26, RAB29, RAB2B, RAB30, RAB33B, RAB34, RAB35, RAB3A, RAB3D, RAB40B, RAB40C, RAB4A, RAB4B, RAB5A, RAB5B, RAB5C, RAB8A, RABL2A, RAC1, RAC2, RAD1, RAD51, RAD9B, RAET1E, RALB, RALGAPA1, RALY, RAMP3, RANBP6, RAPH1, RARRES1, RASGRP2, RASGRP4, RASSF3, RASSF5, RASSF6, RASSF8, RAVER1, RBAK, RBCK1, RBFA, RBM12, RBM14, RBM15, RBM17, RBM22, RBM42, RBM43, RBM45, RBM47, RBSN, RCBTB1, RCBTB2, RCC1, RCC2, RCSD1, RDH12, RDM1, REG4, RELB, RELN, RERGL, REST, RFC3, RFT1, RFX5, RFX8, RGMA, RGMB, RGPD8, RGR, RGS17, RGS20, RGS4, RGS8, RHAG, RHBDD1, RHBDD2, RHBDL2, RHD, RHEB, RHOBTB1, RHOJ, RIC3, RIC8A, RIC8B, RILPL1, RIMKLB, RIN1, RMND5B, RNASEL, RNASET2, RND3, RNF114, RNF135, RNF138, RNF14, RNF141, RNF145, RNF182, RNF185, RNF19B, RNF2, RNF212B, RNF34, RNF41, RNF6, RNF8, RNH1, ROCK2, ROPN1, ROPN1B, RPA2, RPA3, RPL12, RPL14, RPL18, RPL27A, RPL37A, RPL4, RPL5, RPP14, RPP40, RPRD1A, RPS17, RPS21, RPS24, RPS3, RPS3A, RPS6KA4, RPUSD2, RRAS2, RREB1, RRM2, RRP8, RSBN1L, RSPH1, RSPH14, RSPH9, RYR3, SART3, SECISBP2L, SETD5, SGSM2, SHPRH, SIN3B, SKIC2, SLC12A4, SLC12A9, SMARCAD1, SMG7, SNX13, SNX14, SPEF2, SPEG, SPG11, SPTBN1, SRCAP, SSH1, SVEP1, SYCP2, SYNE2, SYNJ1, SYNM, SYNRG, SZT2, TDRD12, TJP1, TLR4, TNS2, TRRAP, TUT1, TYRO3, UACA, UBR4, UBR5, UNC79, UNC80, USH2A, USP33, USPL1, VCAN, VILL, VPS13C, VPS13D, WDR6, WIZ, YTHDC2, YY1AP1, ZBTB20, ZC3H6, ZC3H7A, ZCCHC2, ZFYVE16, ZHX1, ZHX3, ZMYM1, ZMYM6, ZNF208, ZNF226, ZNF268, ZNF280D, ZNF292, ZNF616, ZNF644, ZNF780B, ZNF814, ZNF841, and ZSCAN20;
thereby evaluating or detecting a cancer in a subject.

32. The method of claim 31, wherein at least one of:
a. said evaluating comprises detecting a driver mutation in said cancer;
b. said identifying comprises sequencing said genomic DNA;
c. said identifying comprises deep sequencing or next generation sequencing of said genomic DNA; and
d. said sample is selected from a biopsy and a bodily fluid sample, wherein said bodily fluid comprises cells or cell free DNA.

33. (canceled)

34. (canceled)

35. (canceled)
